(19) United States
(12) Patent Application Publication
(10) Pub. No.: US 2014/0205578 A1
(43) Pub. Date: Jul. 24, 2014

(54) METHODS FOR INCREASING INSULIN SENSITIVITY AND TREATING DIABETES

(71) Applicants: Jonathan D. Powell, Baltimore, MD (US); Bo Xiao, Ellicott City, MD (US); Paul Worley, Baltimore, MD (US); Gregg M. Delgoffe, Memphis, TN (US); Adam Waickman, Elkridge, MD (US)

(72) Inventors: Jonathan D. Powell, Baltimore, MD (US); Bo Xiao, Ellicott City, MD (US); Paul Worley, Baltimore, MD (US); Gregg M. Delgoffe, Memphis, TN (US); Adam Waickman, Elkridge, MD (US)

(73) Assignee: The John Hopkins University, Baltimore, MD (US)

(21) Appl. No.: 14/126,994

(22) PCT Filed: Jun. 15, 2012
(86) PCT No.: PCT/US12/42636

§ 371 Date: Mar. 27, 2014

Related U.S. Application Data
(60) Provisional application No. 61/498,202, filed on Jun. 17, 2011.
(60) Provisional application No. 61/541,686, filed on Sep. 30, 2011.

(51) Int. Cl. C12N 9/64

(52) U.S. Cl. CPC C12N 9/6445
USPC 424/93.21; 424/94.64

(57) ABSTRACT

Described herein are methods for increasing insulin sensitivity and for treating Diabetes (Type I and Type II). Also described herein are methods for increasing the amount of brown fat in a subject and for treating metabolic disorders, including obesity.

### CROSS-REFERENCE TO RELATED APPLICATIONS

This application claims the benefit of U.S. Provisional Application No. 61/498,202, filed on Jun. 17, 2011, and U.S. Provisional Application No. 61/541,686, filed on Sep. 30, 2011; the contents of each of which application is specifically incorporated by reference herein in its entirety.

### BACKGROUND

Diabetes mellitus (“diabetes”) is a chronic metabolic disease characterized by high blood sugar levels caused by either insufficient insulin production (type I diabetes) or a deficient response to insulin that is produced (type II diabetes). Diabetes affects millions of individuals and is a major public health problem throughout the world.

Type I diabetes is characterized by the loss of insulin-producing cells, which leads to insulin deficiency. Type I diabetes is usually the result of a T cell mediated autoimmune attack against pancreatic beta cells and represents the majority of diabetes cases in children. There is no known cure for type I diabetes, and type I diabetics generally manage their symptoms through diet and insulin injections.

The majority of diabetics in the United States suffer from type II diabetes. Type II diabetes is characterized by insulin resistance and is associated with obesity. Type II Diabetics display an array of metabolic disorders which often contribute to the development of cardiovascular disease, stroke, retinopathy, neuropathy, nephropathy and liver disease. Like type I diabetes, there is no known cure for type II diabetes and type II diabetics must manage their symptoms through diet and exercise.

Thus, there is a great need for methods through which increasing insulin sensitivity can be increased in individuals with diabetes. Such methods could be used to restore insulin sensitivity to individuals with type II diabetes and to increase the activity of the low amounts of insulin remaining in many type I diabetics. Such methods would therefore be useful in the treatment of both type I and type II diabetes.

### SUMMARY

In some embodiments, described herein are methods of increasing insulin sensitivity, preventing or treating diabetes (e.g., type I or type II diabetes), increasing brown fat levels, preventing or treating a metabolic disorder (e.g., obesity), promoting weight loss, treating hyperlipidemia and/or treating cardiovascular and vascular disease in a subject. In some embodiments the subject has or is predisposed to diabetes, a metabolic disorder and/or obesity.

In some embodiments the method includes administering to the subject a kallikrein family peptidase or biologically active fragment thereof. In certain embodiments, the kallikrein family peptidase or biologically active fragment thereof has an amino acid sequence that is at least 70% identical to a sequence selected from the group consisting of SEQ ID NO: 1-41 (e.g., SEQ ID NO: 1, 12 or 38).

In some embodiments, the kallikrein family peptidase or biologically active fragment thereof is administered by administering to the subject a pharmaceutical composition that includes a therapeutic amount of an isolated kallikrein family peptidase or biologically active fragment thereof.

In some embodiments the kallikrein family peptidase or biologically active fragment thereof is administered by administering to the subject a population of recombinant cells that expresses the kallikrein family peptidase or a biologically active fragment thereof. In certain embodiments the population of recombinant cells includes T cells, T cell precursors, B cells, B cell precursors, bone marrow stem cells, embryonic stem cells, induced embryonic stem cells, peripheral blood stem cells or a combination thereof. In some embodiments the population of cells is originally derived from the subject.

In certain embodiments the method includes the step of administering to the subject an agent that increases the expression of a Kallikrein family peptidase (e.g., Klk1, Klk2, Klk3, Klk4, Klk5, Klk6, Klk7, Klk8, Klk9, Klk10, Klk11, Klk12, Klk13, Klk14 or Klk15) in the subject. In some embodiments the agent is a small molecule, a polypeptide, an antibody or an inhibitory RNA molecule (e.g., an inhibitory RNA molecule specific for Rheb). In certain embodiments, the subject is administered cells that had been transfected with a nucleic acid molecule that increases the expression of a Kallikrein family peptidase by the cells. In some embodiments, the method includes the step of transfecting the cells.

In some embodiments, the method includes administering to the subject one or more recombinant cells that have reduced Rheb expression or activity (e.g. the one or more cells have reduced Rheb expression or activity compared to a non-recombinant cell of the same type and/or from the same species). In certain embodiments, a Rheb gene or a Rheb promoter is mutated or knocked out in the one or more recombinant cells. In some embodiments, both Rheb genes or Rheb promoters are mutated or knocked out in the one or more recombinant cells. In some embodiments, the one or more recombinant cells express a Rheb-specific inhibitory RNA molecule (e.g., a siRNA molecule, an shRNA molecule or a micro-RNA molecule).

In some embodiments, the one or more recombinant cells include T cells, T cell precursors, B cells, B cell precursors, bone marrow stem cells, embryonic stem cells, induced embryonic stem cells, peripheral blood stem cells or a combination thereof. In certain embodiments, the one or more recombinant cells are syngeneic to the subject. In some embodiments, the one or more recombinant cells were generated from one or more non-recombinant cells of the subject.

In certain embodiments, the method also includes the step of harvesting the one or more non-recombinant cells from the subject and/or the step of converting the one or more non-recombinant cells into the one or more recombinant cells. In some embodiments converting is performed by mutating a Rheb gene or a Rheb promoter in the one or more non-recombinant cells (e.g., using standard recombinant techniques known in the art), thereby generating one or more recombinant cells having reduced Rheb expression. In some embodiments, the step of converting is performed by introducing into the one or more non-recombinant cells a Rheb specific inhibitory RNA molecule expression vector. In some embodiments, described herein are methods for determining whether a test compound is a likely therapeutic agent for increasing insulin sensitivity, treating diabetes (e.g., type I diabetes or type II diabetes), increasing brown fat levels and/or treating a metabolic disorder (e.g., obesity).

In some embodiments the methods include the steps of (a) contacting a cell (e.g., a mouse cell or a human cell, such as a T cell) with the test compound and (b) detecting the expression of a Kallikrein family peptidase (e.g., Klk1b22, Klk1 or Klk12) by the cell, where a test compound that causes increased expression of a Kallikrein family peptidase is a likely therapeutic agent. In certain embodiments the expression of the Kallikrein family peptidase is detected by detecting Kallikrein family peptidase mRNA. In some embodiments the expression of the Kallikrein family peptidase is detected by detecting Kallikrein family peptidase protein. In some embodiments the Kallikrein family peptidase is linked to a detectable moiety and the expression of the Kallikrein family peptidase is detected by detecting the detectable moiety.

In some embodiments the methods include the steps of (a) contacting a cell (e.g., a human cell or a mouse cell, such as a T cell) with the test compound, wherein the cell comprises a nucleic acid sequence encoding a detectable moiety operably linked to a promoter of a Kallikrein family peptidase gene (e.g., a Klk1b22, Klk1 or Klk12 promoter), and (b) detecting the expression of the detectable moiety by the cell, where a test compound that causes increased expression of the detectable moiety is a likely therapeutic agent.

### BRIEF DESCRIPTION OF THE DRAWINGS

FIG. 1 shows the median weight of Rheb^{fl/fl} CD4cre mice and wildtype (WT) mice.

FIG. 2 shows a body fat analysis of Rheb^{fl/fl} CD4cre mice and wildtype mice.

FIG. 3 shows the glucose uptake and insulin sensitivity of Rheb^{fl/fl} CD4cre mice and wildtype mice.

FIG. 4 shows the serum triglyceride and serum High-density lipoprotein levels of Rheb^{fl/fl} CD4cre mice and wildtype mice.

FIG. 5 shows the food consumption of Rheb^{fl/fl} CD4cre mice and wildtype mice.

FIG. 6 shows the tissue uptake of glucose in Rheb^{fl/fl} CD4cre mice and wildtype mice as determined using 18-F-FDG Proton Emission Tomography imaging.

FIG. 7 shows the expression of BMP7, fibroblast Growth Factor-21 and Acyl-CoA thioesterase in the livers of Rheb^{fl/fl} CD4cre mice and wildtype mice.

FIG. 8 shows the L-lactate production of T cells isolated from either Rheb^{fl/fl} CD4cre mice or wildtype mice.

FIG. 9 shows the respiratory exchange ratio of Rheb^{fl/fl} CD4cre mice and wildtype mice.

FIG. 10 shows the change in body weight of irradiated wildtype mice that had undergone a bone marrow transplant of bone marrow isolated from either Rheb^{fl/fl} CD4cre mice or wildtype mice.

FIG. 11 shows the glucose uptake of RAG^{fl/fl} mice that had received a T cell transplant from either Rheb^{fl/fl} CD4cre mice or wildtype mice.

FIG. 12 shows the insulin sensitivity of diabetic mice both before and after receiving T cells from either Rheb^{fl/fl} CD4cre mice or wildtype mice.

FIG. 13 shows the glucose uptake of wildtype mice after administration of serum taken from either Rheb^{fl/fl} CD4cre mice or wildtype mice.

FIG. 14 shows the amino acid sequences of human Kallikrein family peptidases.

FIG. 15 shows the amino acid sequences of mouse Kallikrein family peptidases.

FIG. 16 shows the nucleic acid sequences of human Kallikrein family peptidases.

FIG. 17 shows the nucleic acid sequences of mouse Kallikrein family peptidases.

FIGS. 18A-18B show enhancement of both insulin receptor phosphorylation

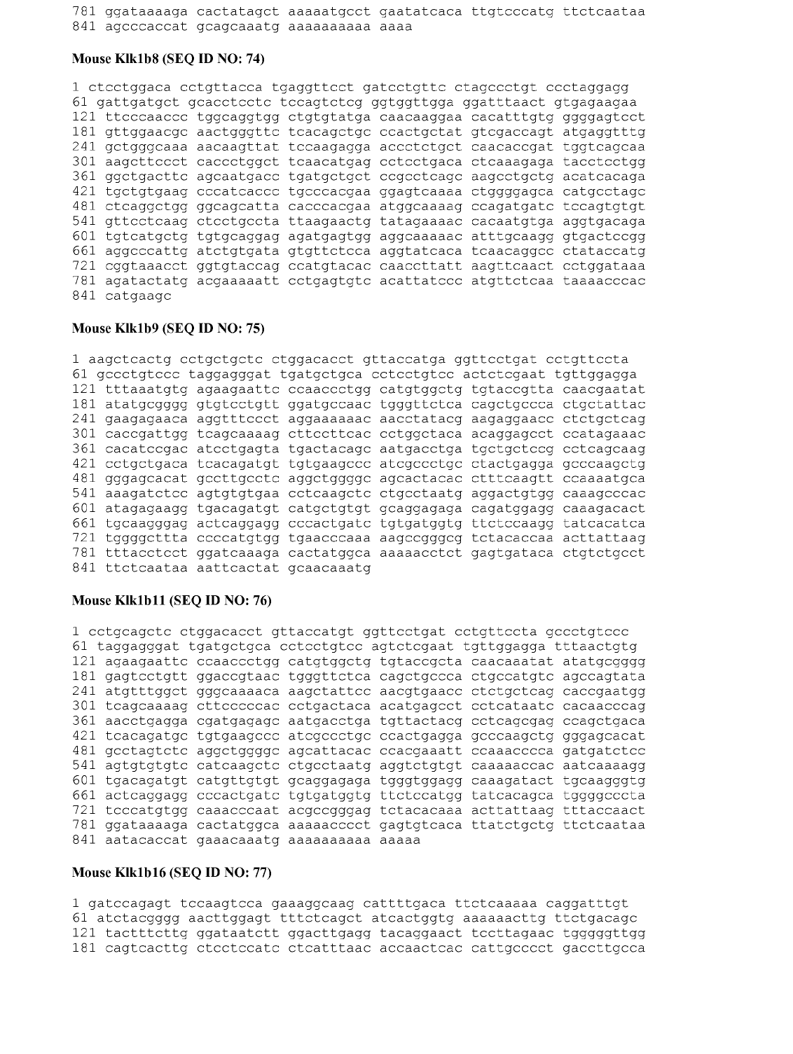

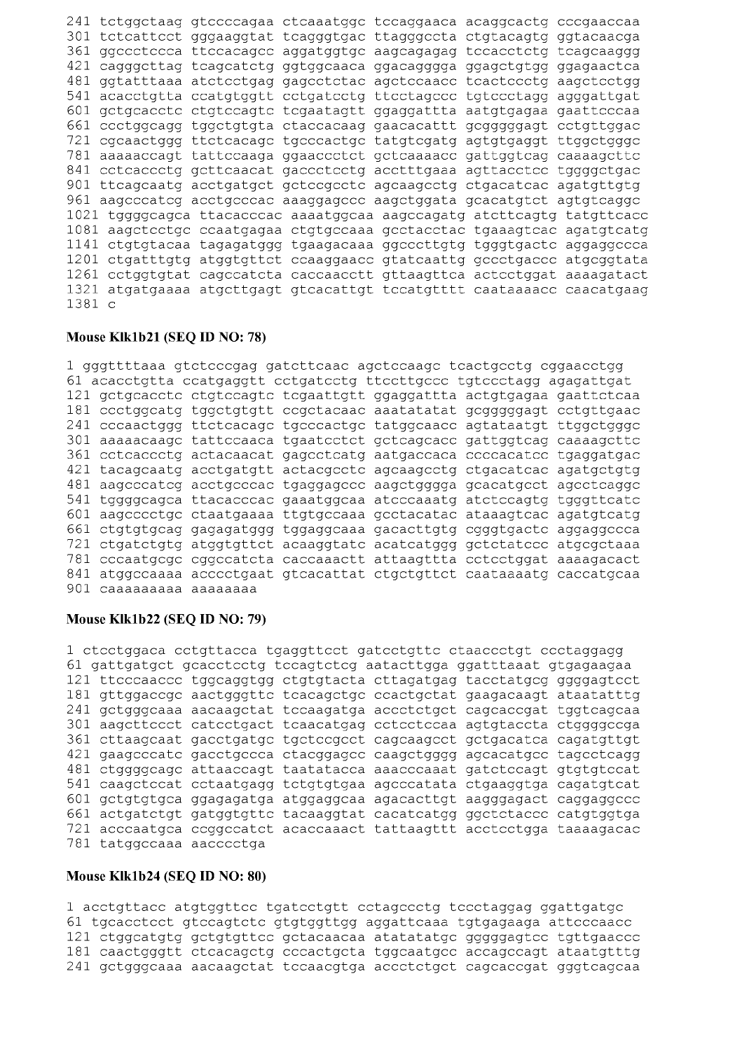

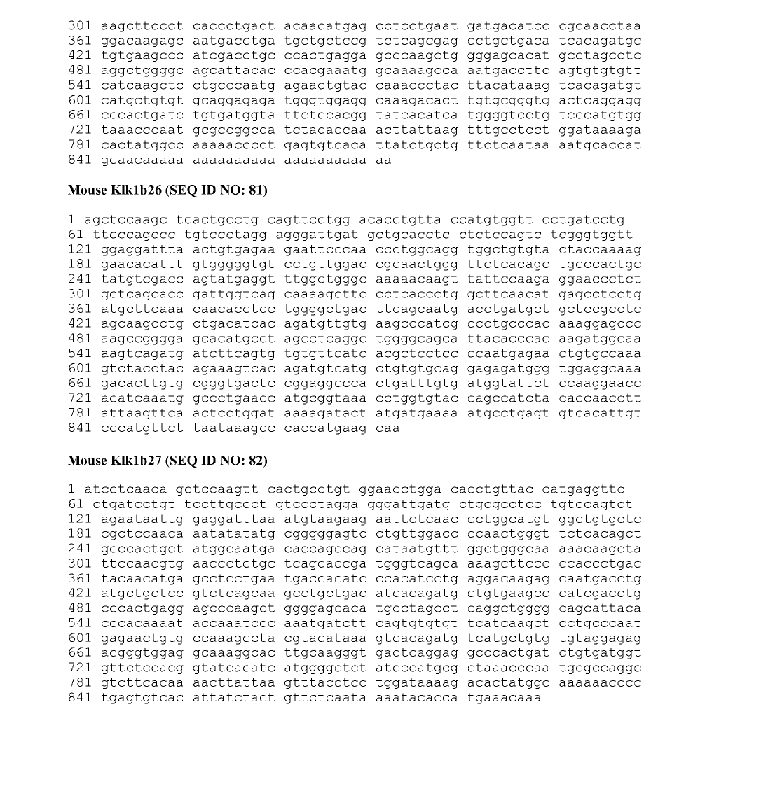

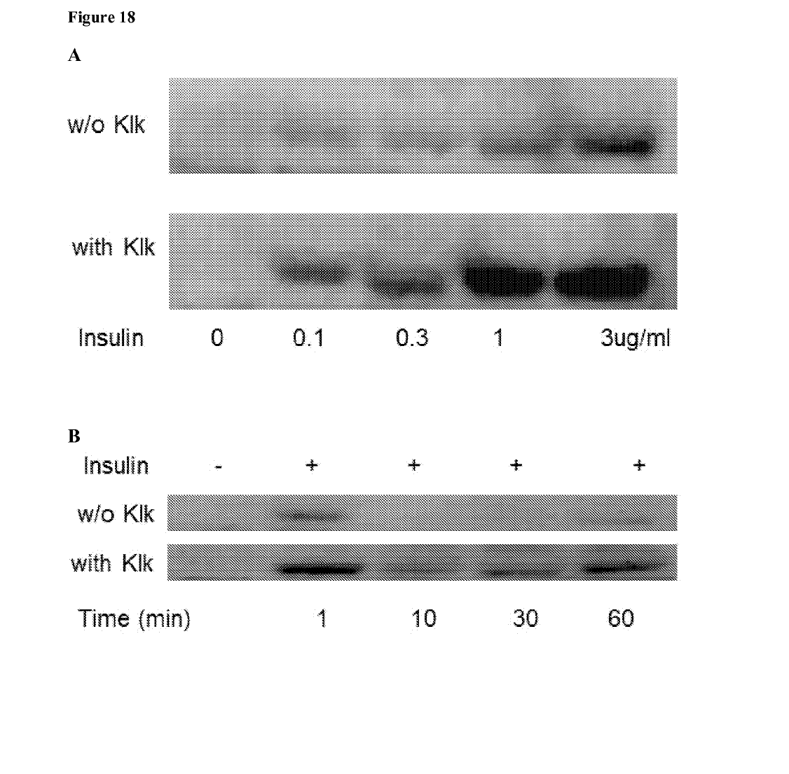

(FIG. 18A) and prolonged insulin receptor phosphorylation (FIG. 18B) by Klk1b22 in response to a dose response of insulin.

### DETAILED DESCRIPTION

### I. General

Described herein are compositions and methods for increasing insulin sensitivity and/or brown fat levels in a subject and for treating and preventing a wide range of diseases and disorders, including diabetes, metabolic disorders and obesity. Also described herein are compositions and methods for the identification of additional therapeutic agents that can be used in the methods described herein.

Diabetes is a significant health problem throughout the world. Diabetics are unable to regulate their blood glucose levels either because of reduced insulin production (type I diabetes) or because of reduced insulin sensitivity (type II diabetes). Serious long-term complications associated with diabetes include cardiovascular disease, chronic renal failure and retinal damage.

In certain embodiments, provided herein are methods and compositions for increasing insulin sensitivity. Such methods and compositions are useful for the treatment of both type I diabetes and type II diabetes. For example, treatment of type I diabetics using the compositions and methods described herein enhances the potency of the low levels of insulin produced by the diabetic subject, while treatment of type II diabetics restores insulin sensitivity to their cells. Furthermore, the compositions and methods described herein can be combined with the administration of insulin or insulin analogs in order to increase insulin's efficacy and reduce the dose required to achieve a therapeutic effect.

In some embodiments, a therapeutic effect (e.g., increased insulin sensitivity, prevention and/or treatment of diabetes, increased brown fat levels, prevention and/or treatment of a metabolic disorder, prevention and/or treatment of obesity, increased weight-loss, prevention and/or treatment of hyperlipidemia, treatment and/or prevention of cardiovascular and vascular disease) is achieved in a subject through the administration of a kallikrein family peptidase or biologically active fragment thereof In other embodiments, a therapeutic effect is achieved through the administration of a therapeutic agent that enhances the expression or activity of a kallikrein family peptidase.

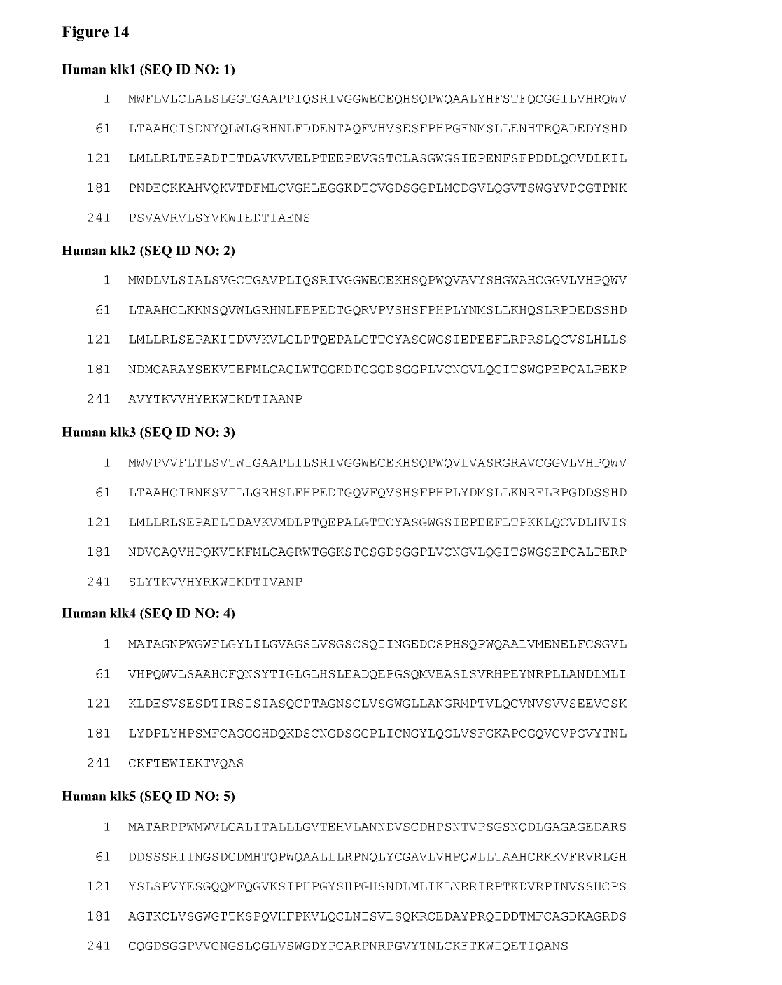

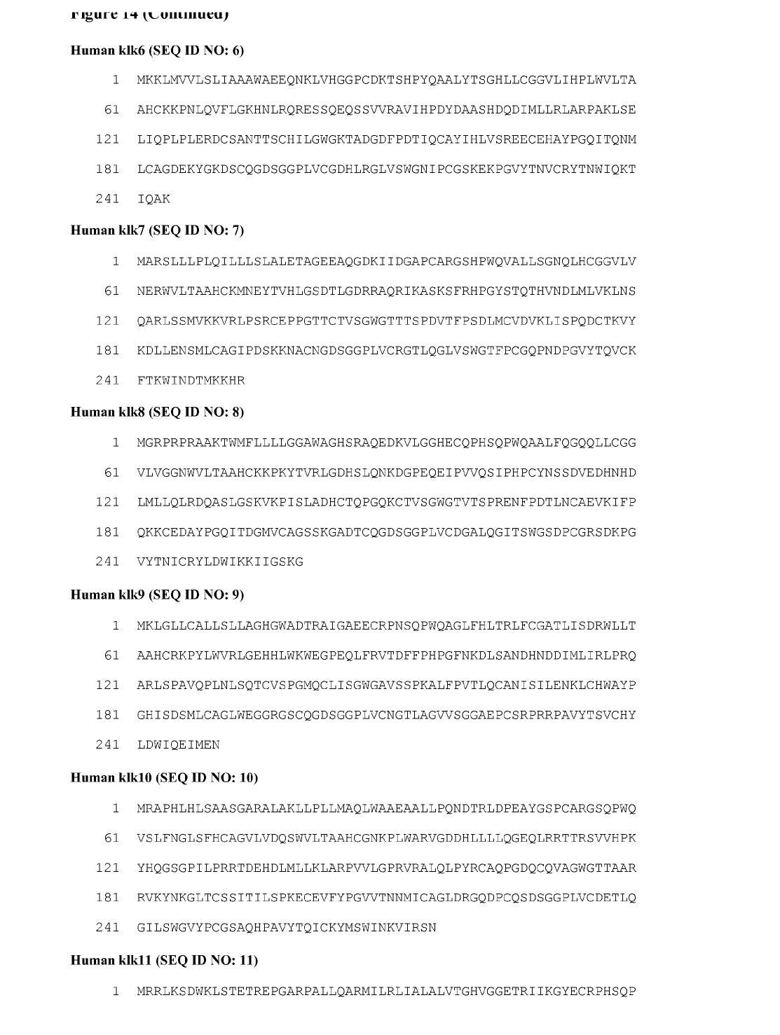

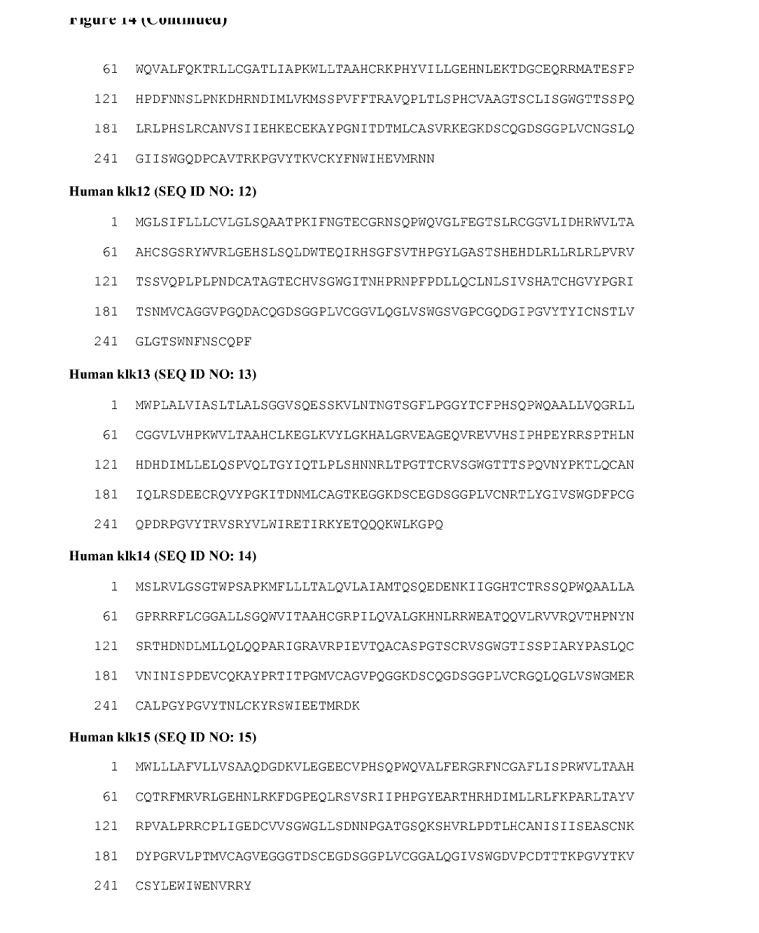

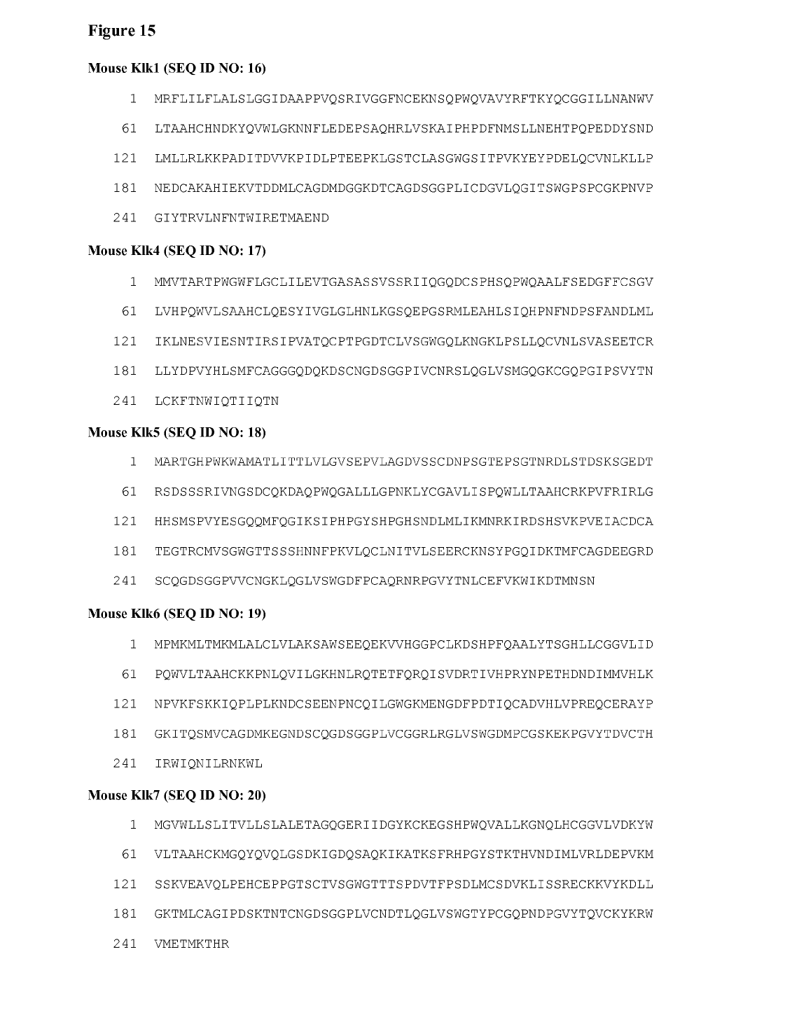

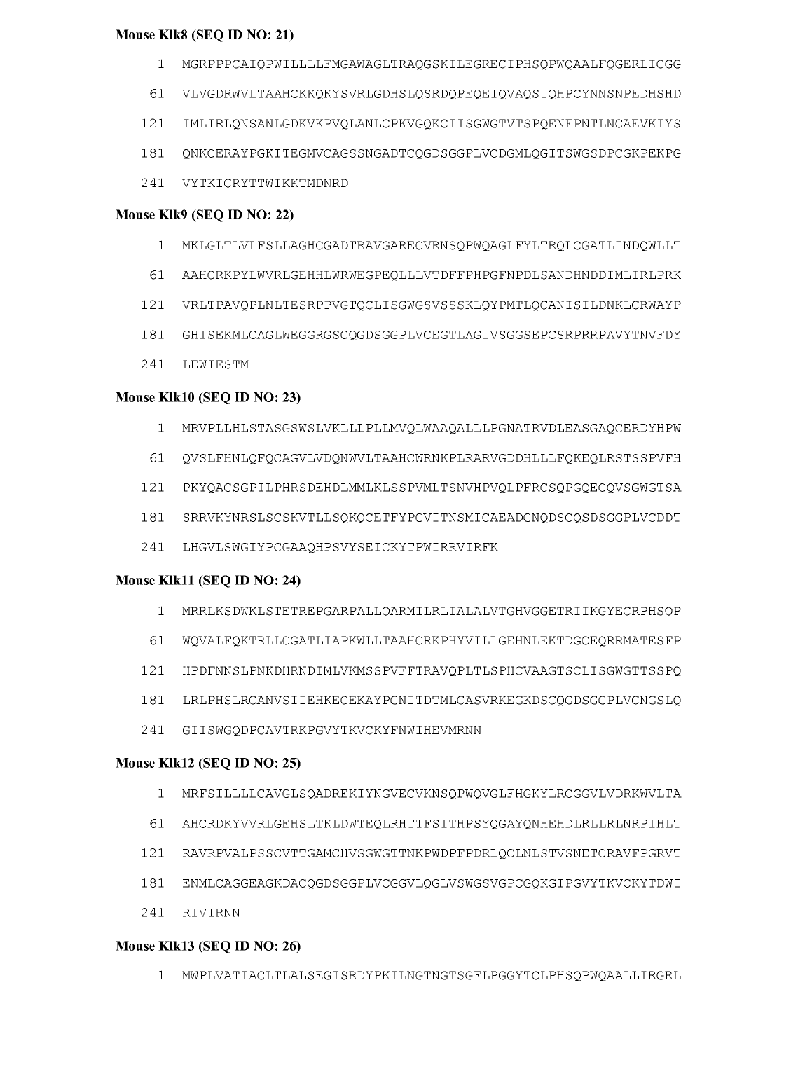

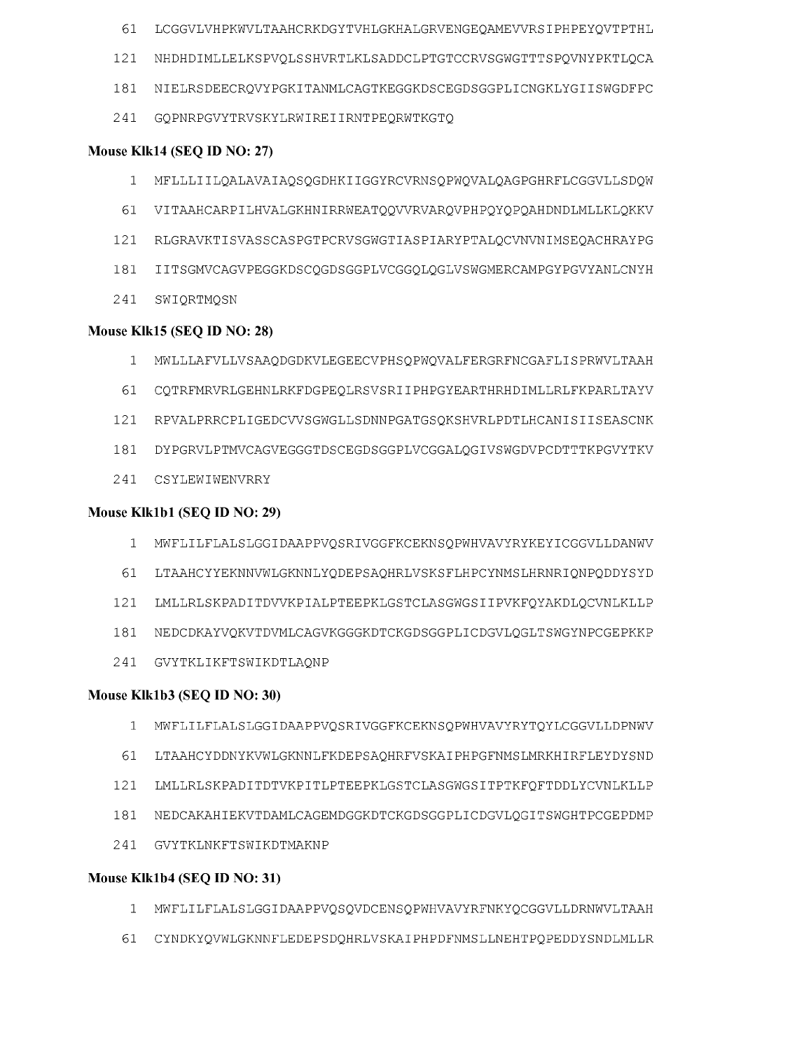

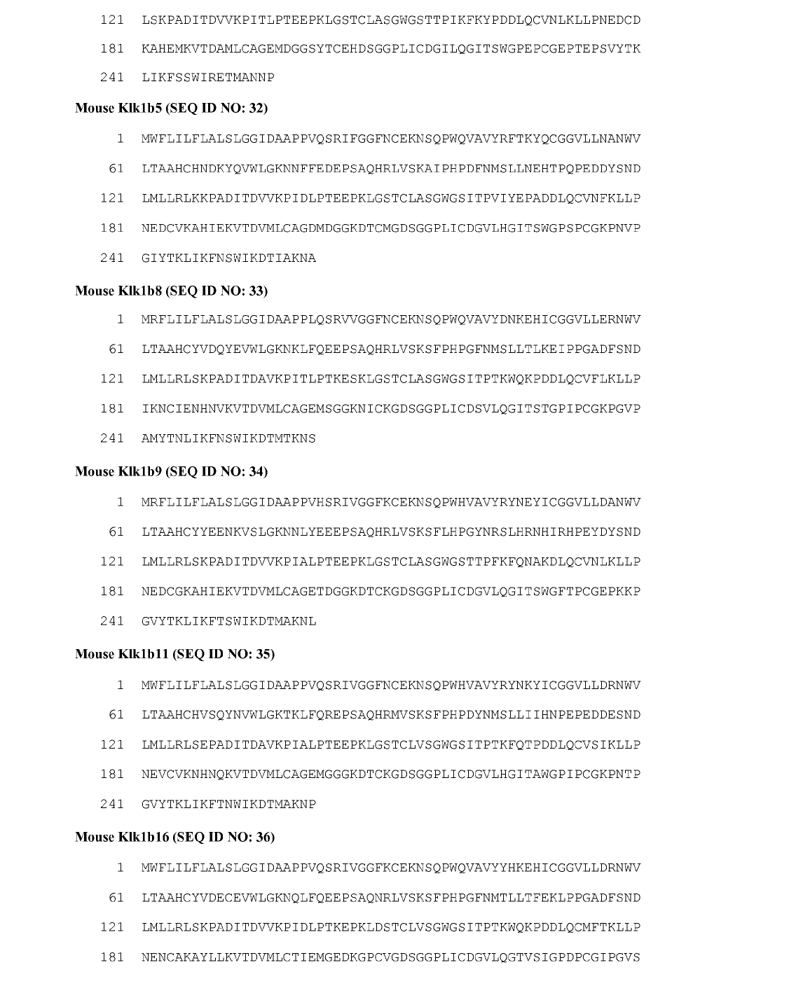

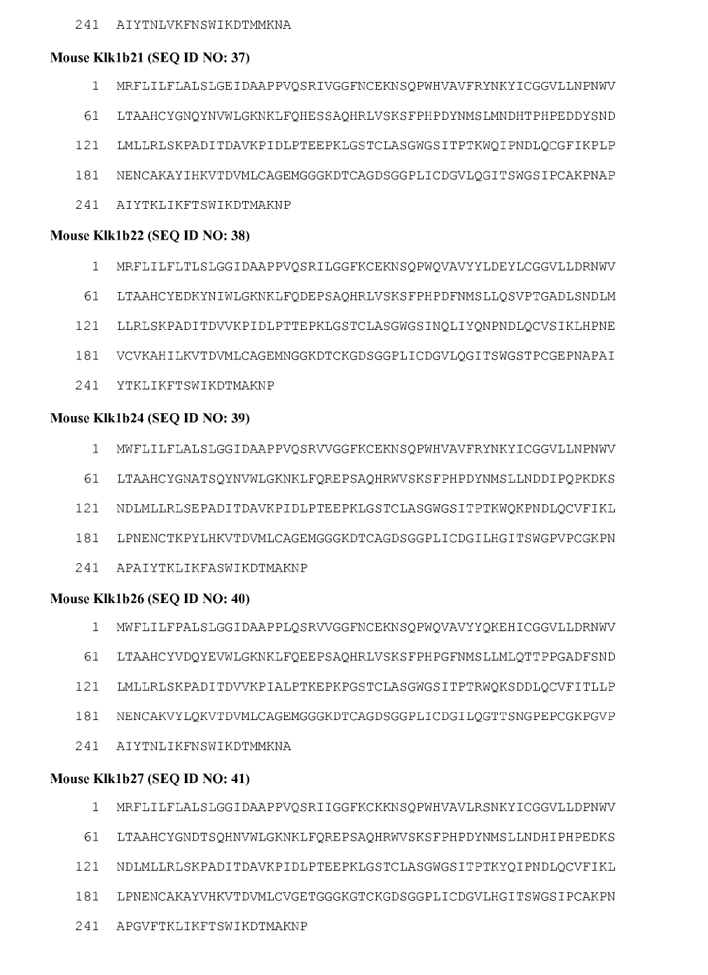

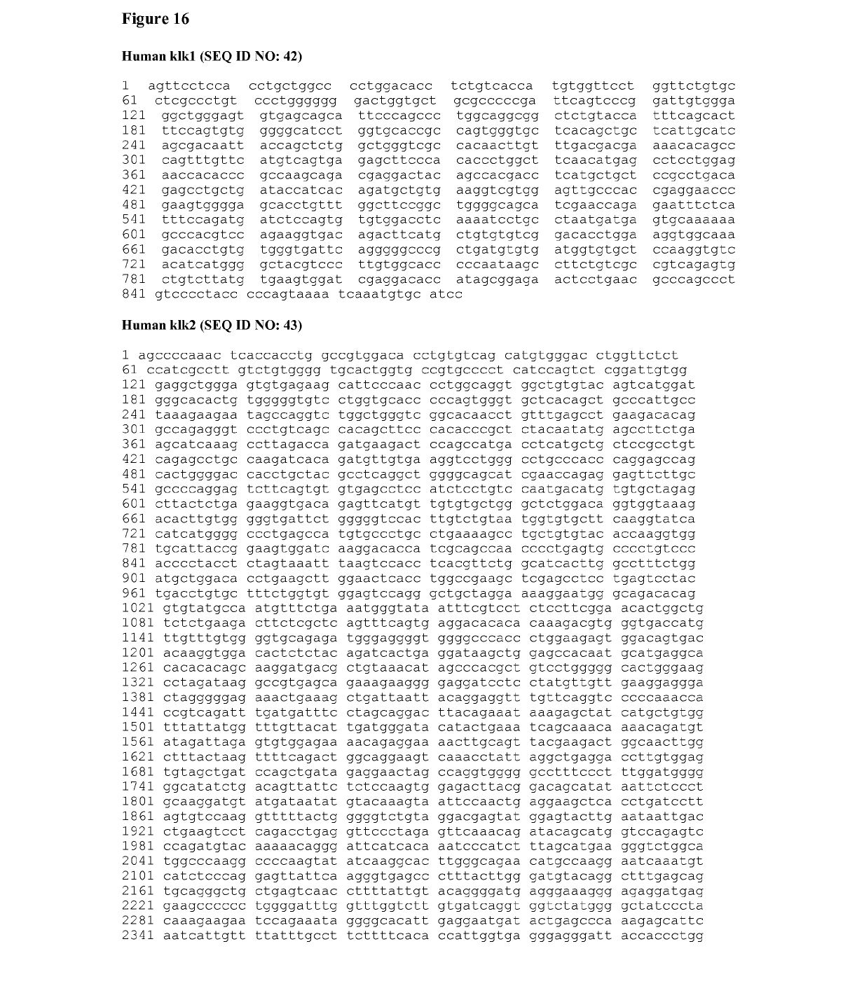

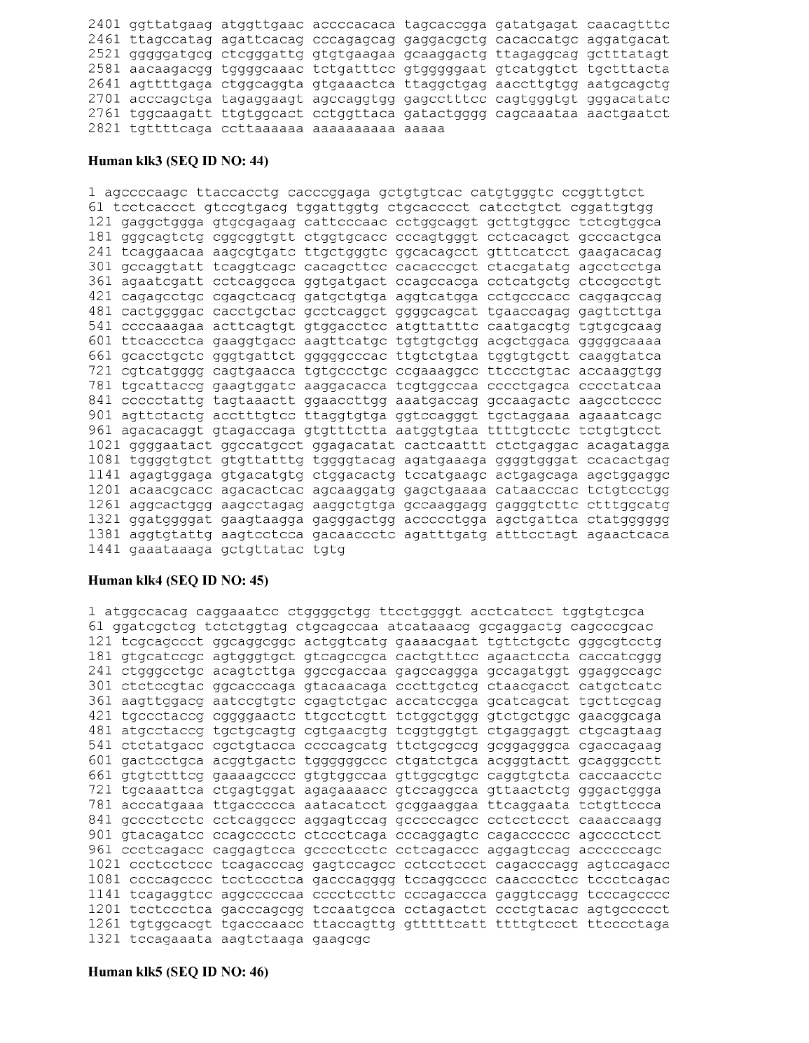

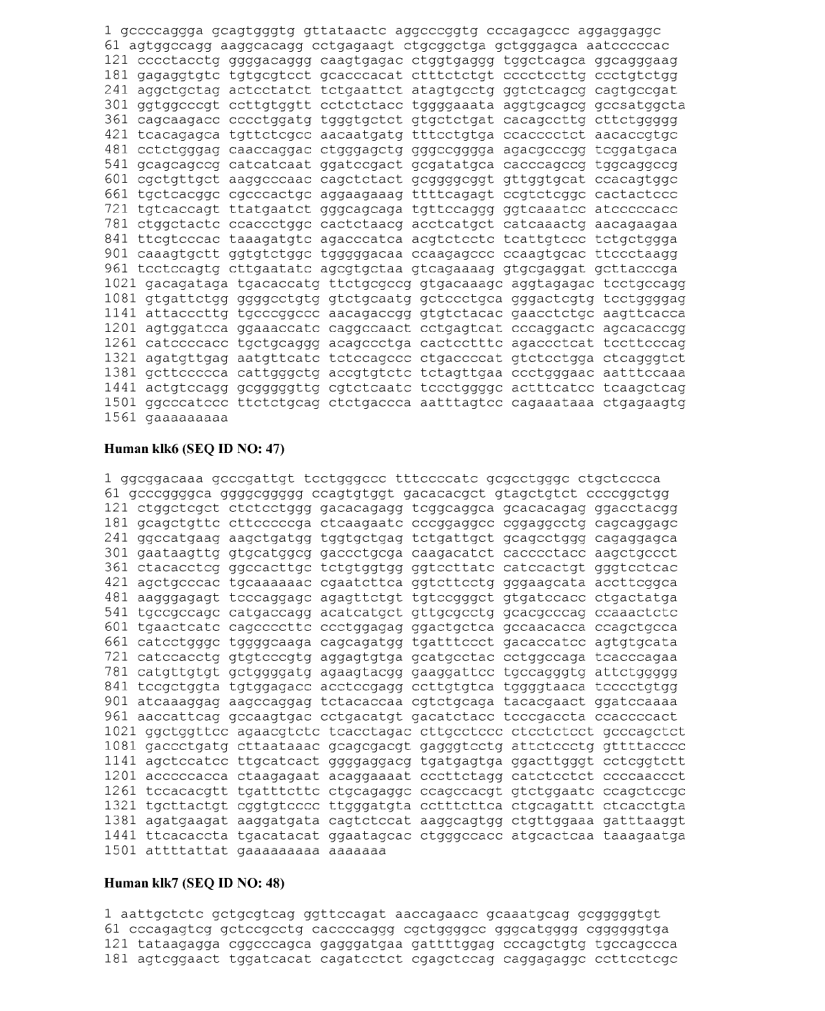

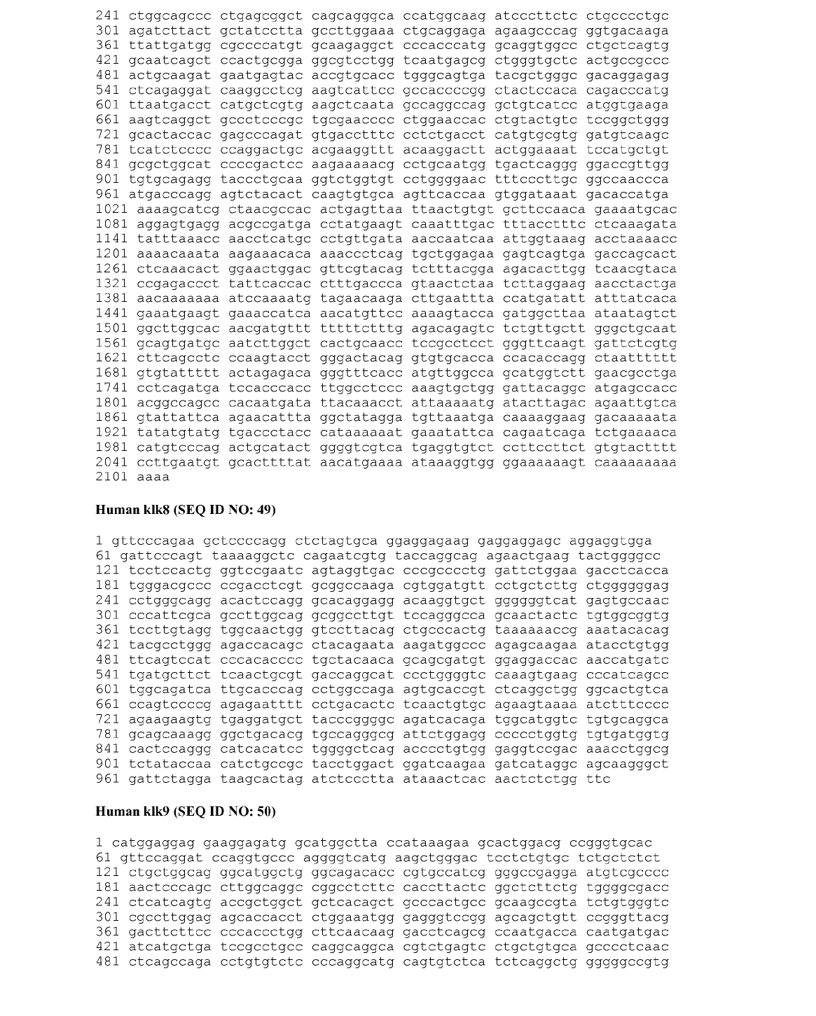

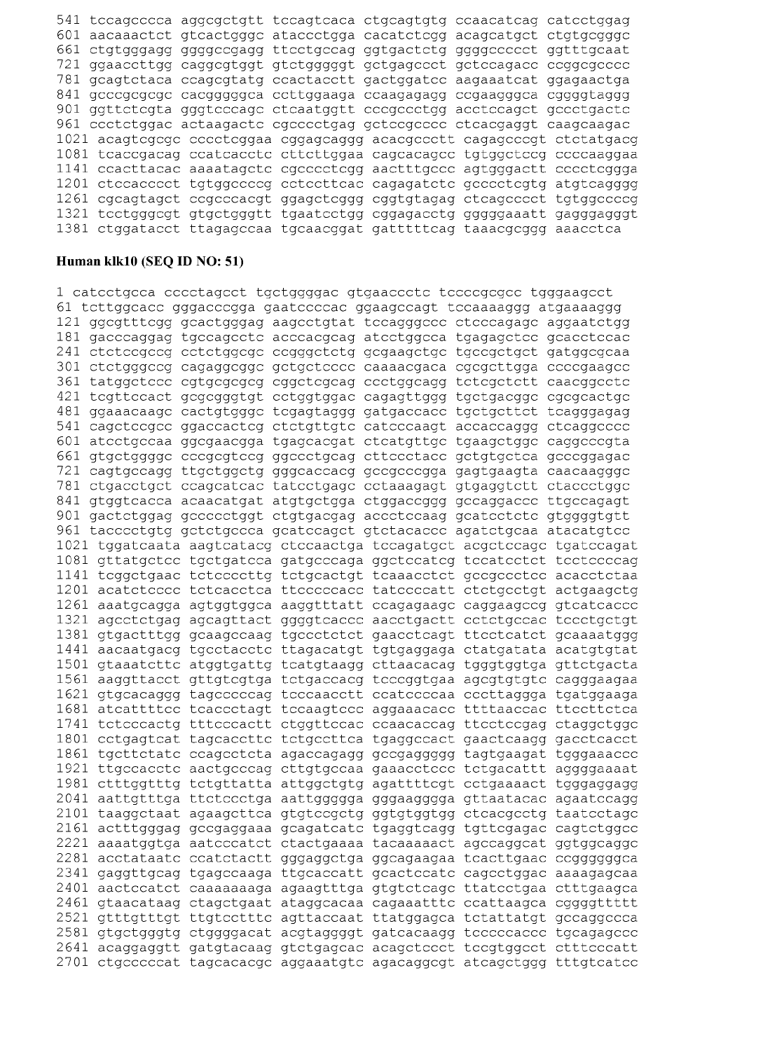

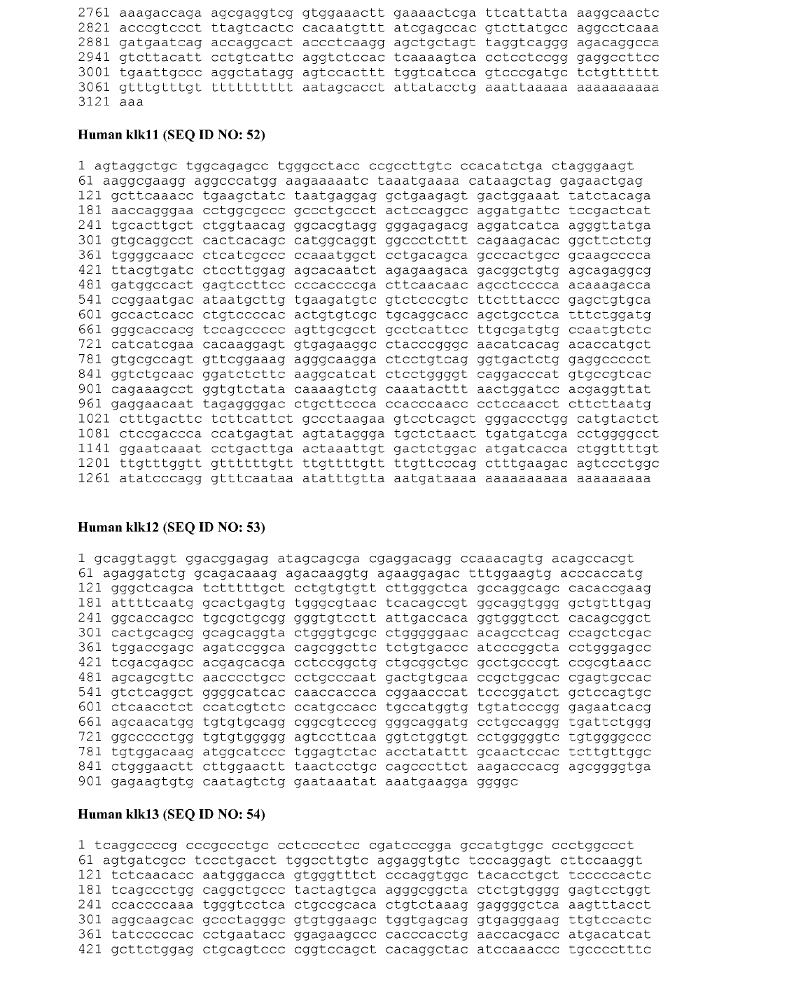

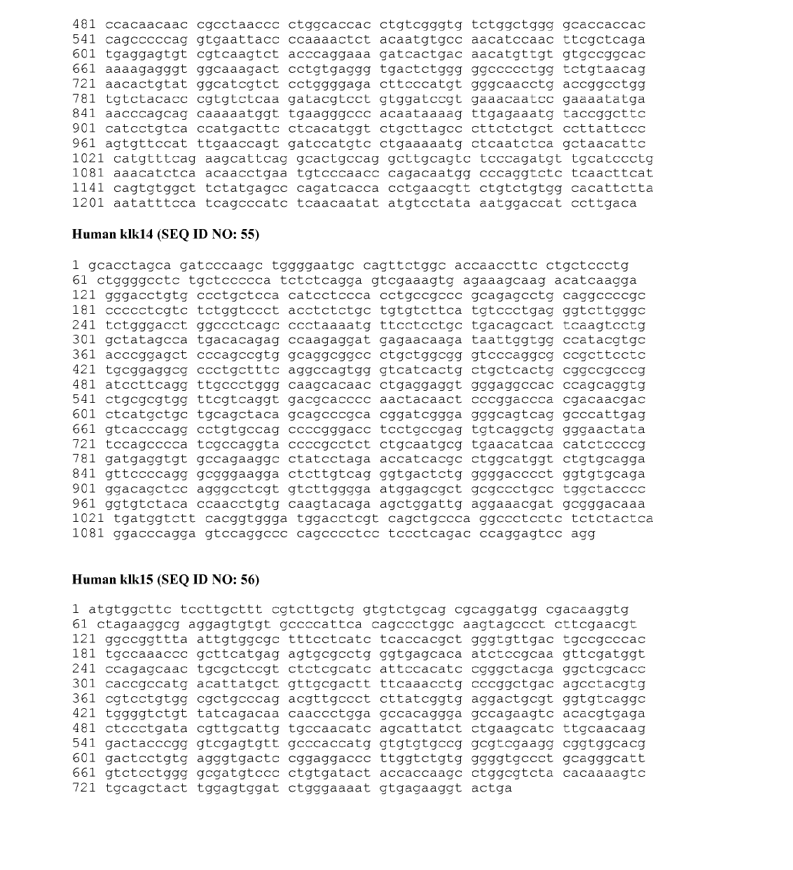

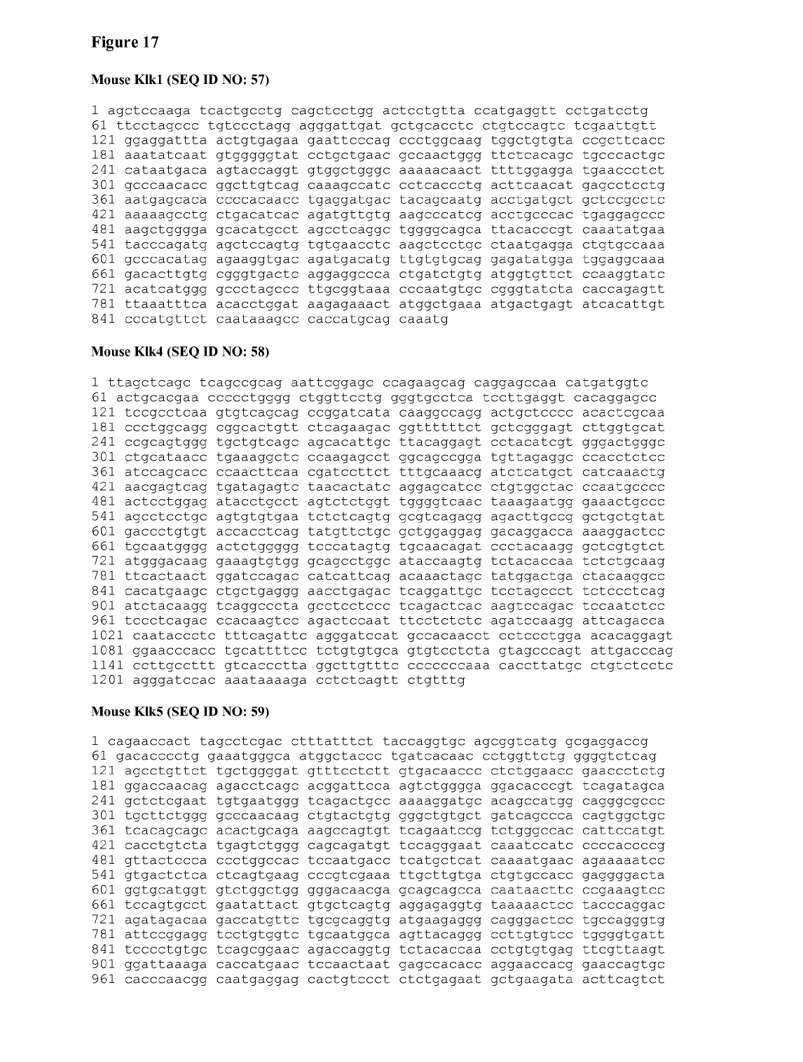

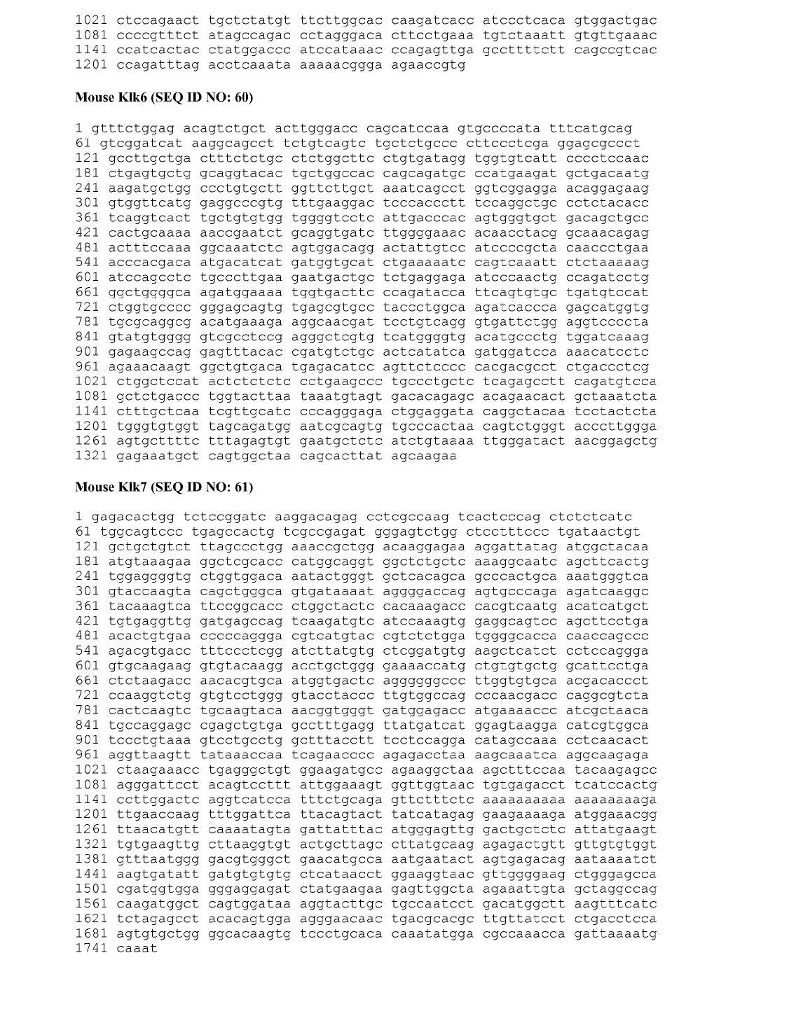

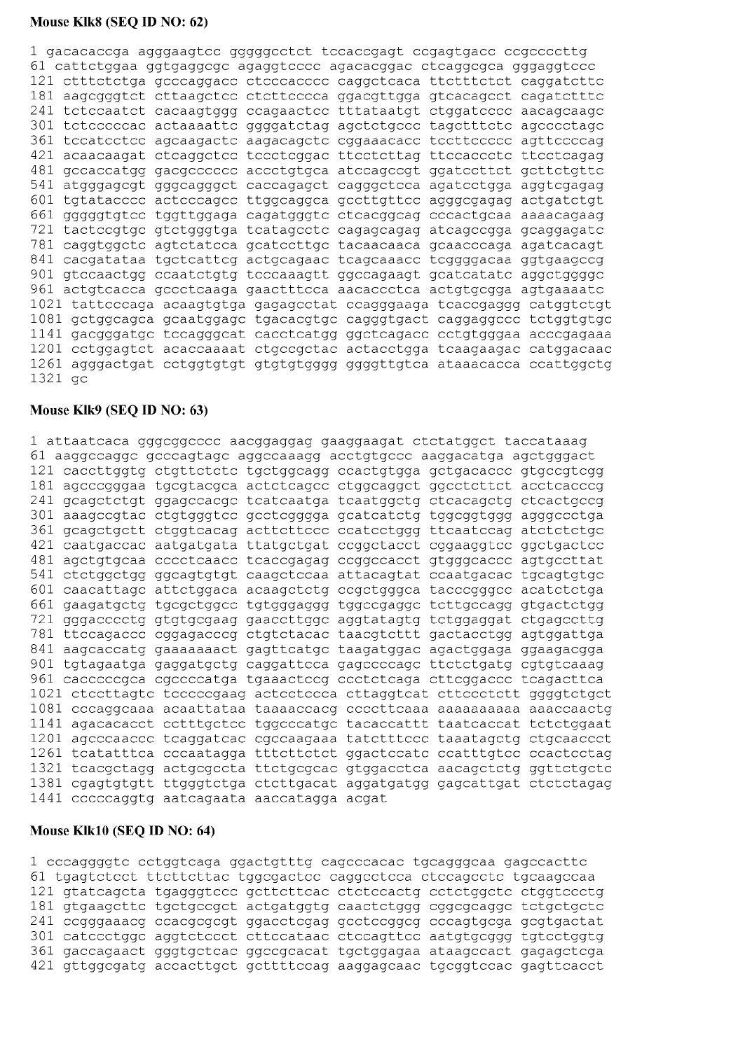

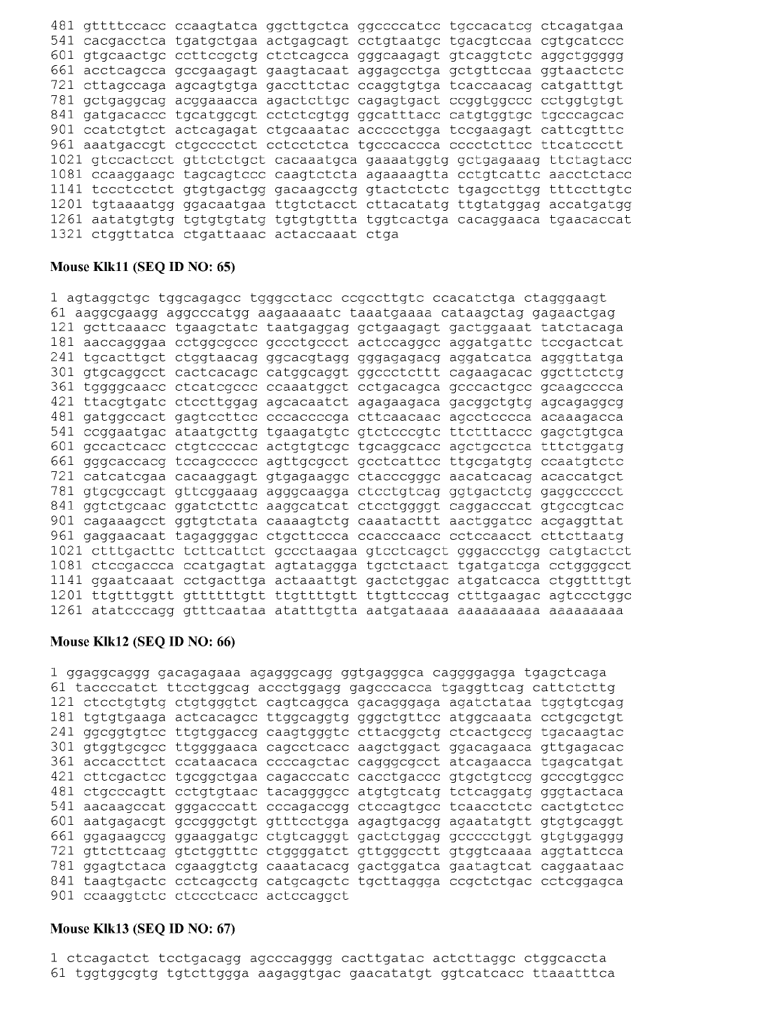

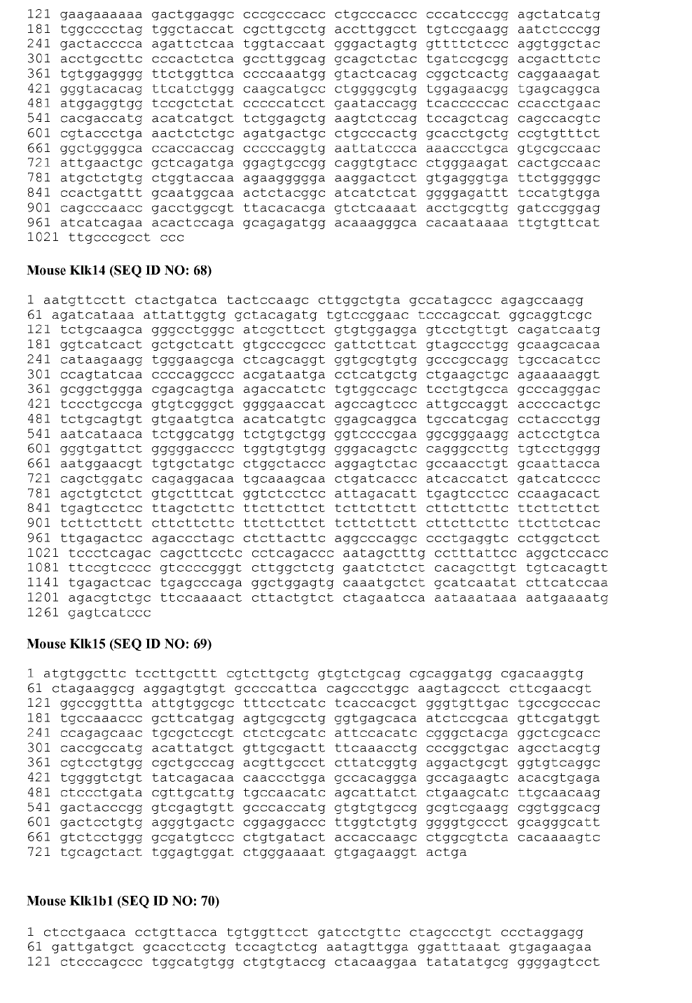

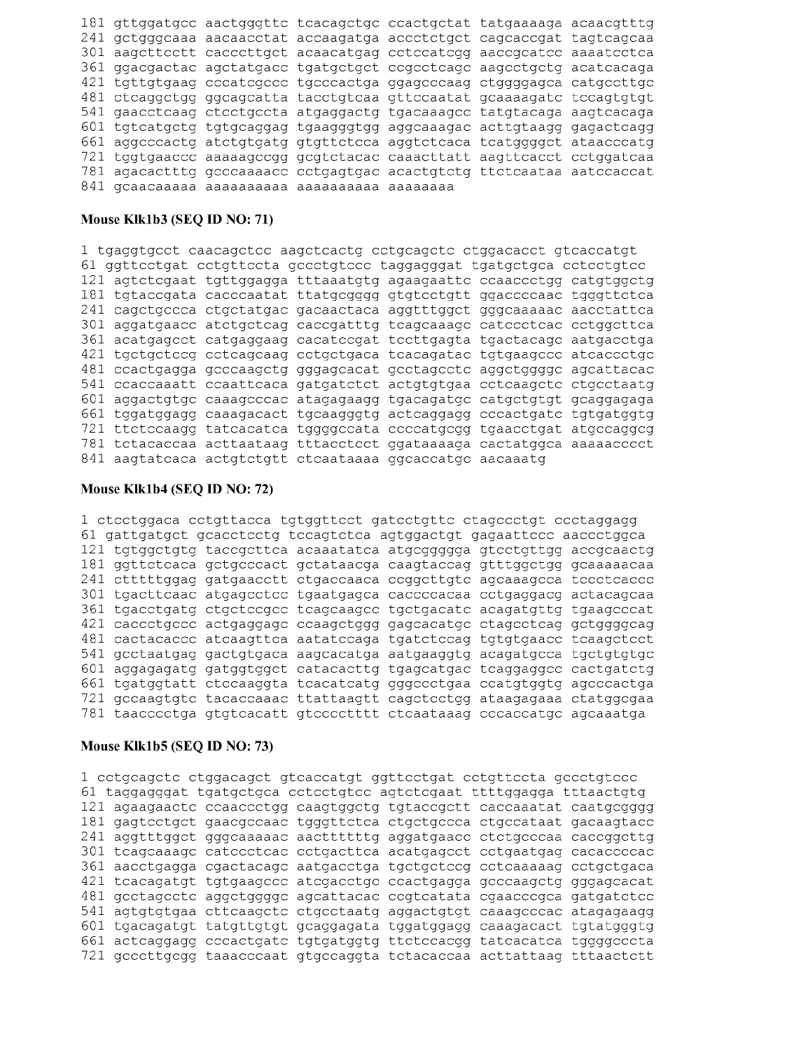

The kallikrein family of peptidases is a multigene family of highly conserved serine proteases. The amino acid sequences of human kallikrein family peptidases are provided in SEQ ID NO: 1-15 and FIG. 14. The amino acid sequences of mouse kallikrein family peptidases are provided in SEQ ID NO: 16-41 and FIG. 15. The nucleic acid sequences of human kallikrein family peptidases are provided in SEQ ID NO: 42-56 and FIG. 16. The nucleic acid sequences of mouse kallikrein family peptidases are provided in SEQ ID NO: 57-82 and FIG. 17. As described herein, increased levels of a kallikrein family peptidase in a subject results in increased insulin sensitivity, increased glucose uptake and increased levels of brown fat. As such, administration of a kallikrein family peptidase or an agent that increases the expression or activity of a kallikrein family peptidase is useful in the treatment of diabetes and metabolic disorders, including obesity.

In some embodiments, a therapeutic effect (e.g., increased insulin sensitivity, prevention and/or treatment of diabetes, increased brown fat levels, prevention and/or treatment of a metabolic disorder, prevention and/or treatment of obesity, increased weight-loss, prevention and/or treatment of hyperlipidemia, treatment and/or prevention of cardiovascular and vascular disease) is achieved in a subject through the administration of one or more recombinant cells (e.g., lymphocytes, such as T cells, or lymphocyte precursors, such as hematopoietic stem cells) that have reduced Rheb expression or activity. Rheb is a small GTPase member of the mTOR signaling pathway that plays a critical role in regulating cell proliferation and survival. The human Rheb mRNA sequence is provided at GI:100913214, while the human Rheb protein sequence is provided at GI:5032041, each of which is incorporated by reference. The mouse Rheb mRNA sequence is provided at GI:133893211, while the mouse Rheb protein sequence is provided at GI:28626508, each of which is incorporated by reference.

### II. Definitions

In order that the present invention may be more readily understood, certain terms are first defined. Additional definitions are set forth throughout the detailed description.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent (e.g. a kallikrein family peptidase or an agent that increases the expression or activity of a kallikrein family peptidase) or composition to a subject, and includes, but is not limited to, administering by a medical professional and self-administering.

The term “agent” is used herein to denote a chemical compound, a small molecule, a mixture of chemical compounds, a biological macromolecule (such as a nucleic acid, an antibody, a protein or portion thereof), or an extract made from biological materials such as bacteria, plants, fungi, or animal (particularly mammalian) cells or tissues. Agents may be identified as having a particular activity (e.g. enhancement of insulin sensitivity) by screening assays described herein below. The activity of such agents may render them suitable as a “therapeutic agent” which is a biologically, physiologically, or pharmacologically active substance (or substances) that acts locally or systemically in a subject.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The term “biologically active fragment” refers to a fragment of a kallikrein family peptidase that retains at least a portion of the biological activity of the entire kallikrein family peptidase, such as the ability to enhance insulin sensitivity.

As used herein, the term “diabetes” refers to a number of well-known conditions. Insulin resistance is defined as a state in which circulating insulin levels in excess of the normal response to a glucose load are required to maintain the euglycemic state (Ford E S, et al. JAMA. (2002) 287:356-9, which is expressly incorporated by reference). Insulin resistance, and the response of a subject with insulin resistance to therapy, may be quantified by assessing the homeostasis model assessment to insulin resistance (HOMA-IR) score, a reliable indicator of insulin resistance (Katsuki A, et al. Diabetes Care 2001; 24:362-5, which is expressly incorporated by reference). The estimate of insulin resistance by the homeostasis assessment model (HOMA)-IR score is calculated with the formula (Galvin P, et al. Diabet Med 1992;9:921-8): HOMA-IR=[fasting serum insulin (μU/mL)]×[fasting plasma glucose (mmol/L)/22.5]. Subjects with a predisposition for the development of impaired glucose tolerance (IGT) or type 2 diabetes are those having euglycemia with hyperinsulinemia are by definition, insulin resistant. A typical subject with insulin resistance is usually overweight or obese. The term “pre-diabetes” is the condition wherein an individual is pre-disposed to the development of type 2 diabetes. Pre-diabetes extends the definition of impaired glucose tolerance to include individuals with a fasting blood glucose within the high normal range 100 mg/dL (J. B. Meigs, et al. Diabetes 2003; 52:1475-1484, which is expressly incorporated by reference) and fasting hyperinsulinemia (elevated plasma insulin concentration). The scientific and medical basis for identifying pre-diabetes as a serious health threat is laid out in a Position Statement entitled “The Prevention or Delay of Type 2 Diabetes” issued jointly by the American Diabetes Association and the National Institute of Diabetes and Digestive and Kidney Diseases (Diabetes Care 2002; 25:742-749, which is expressly incorporated by reference). Individuals likely to have insulin resistance are those who have two or more of the following attributes: 1) overweight or obese, 2) high blood pressure, 3) hyperlipidemia, 4) one or more 1^{st} degree relative with a diagnosis of IGT or type 2 diabetes. Insulin resistance can be confirmed in these individuals by calculating HOMA-IR score. Insulin resistance may be defined as the clinical condition in which an individual has a HOMA-IR score>4.0 or a HOMA-IR score above the upper limit of normal as defined for the laboratory performing the glucose and insulin assays. Type 2 diabetes is defined as the condition in which a subject has a fasting blood glucose or serum glucose concentration greater than 125 mg/dl (6.94 mmol/L). In addition, treating, preventing, or diagnosing diabetes can be measured or achieved in some embodiments of the present invention according to measurements of insulin receptor phosphorylation modulation (e.g., increased insulin receptor phosphorylation) and/or prolonged insulin receptor phosphorylation.

The term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The terms “metabolic disorder” include a disorder, disease or condition which is caused or characterized by an abnormal metabolism (i.e., the chemical changes in living cells by which energy is provided for vital processes and activities) in a subject. Metabolic disorders include diseases, disorders, or conditions associated with aberrant thermogenesis or aberrant adipose cell (e.g., brown or white adipose cell) content or function. Metabolic disorders can detrimentally affect cellular functions such as cellular proliferation, growth, differentiation, or migration, cellular regulation of homeostasis, inter- or intra-cellular communication; tissue function, such as liver function, muscle function, or adipocyte function; systemic responses in an organism, such as hormonal responses (e.g., insulin response).

Examples of metabolic disorders include obesity, including insulin resistant obesity, diabetes, noninsulin dependent diabetes mellitus (NIDDM or Type II diabetes), insulin dependent diabetes mellitus (IDDM or Type I diabetes), type II diabetes, insulin resistance such as impaired glucose tolerance, glucose intolerance, atherosclerosis, atheromatous disease, heart disease, hypertension, stroke, Syndrome X, hyperphagia, endocrine abnormalities, triglyceride storage disease, Bardet-Biedl syndrome, Lawrence-Moon syndrome, Prader-Labhart-Willi syndrome, Werner's syndrome, dysfunctions associated with lipid biosynthesis, lipid transport, triglyceride levels, plasma levels, and plasma cholesterol, dyslipidemias associated with hyperlipidemia, elevated free fatty acids, hypercholesterolemia, hypertriglyceridemia, elevated low density lipoprotein-(LDL)-cholesterol, elevated very low density lipoprotein-(VLDL)-cholesterol, elevated intermediate density lipoprotein-(IDL)-cholesterol, or reduced high density lipoprotein-(HDL)-cholesterol. A metabolic disorder (e.g., diabetes and/or obesity) is “treated” if at least one symptom of the metabolic disorder (e.g., diabetes and/or obesity) is alleviated, terminated, slowed, or prevented. As used herein, a metabolic disorder (e.g., diabetes and/or obesity) is also “treated” if recurrence or metastasis of the metabolic disorder (e.g., diabetes and/or obesity) is reduced, slowed, delayed, or prevented.

In addition, metabolic disorders are associated with one or more discrete phenotypes. For example, body mass index (BMI) of a subject is defined as the weight in kilograms divided by the square of the height in meters, such that BMI has units of kg/m².

In some embodiments, obesity is defined as the condition wherein the individual has a BMI equal to or greater than 30 kg/m². In another aspect, the term obesity is used to mean visceral obesity which can be defined in some embodiments as a waist-to-hip ratio of 1.0 in men and 0.8 in women, which, in another aspect defines the risk for insulin resistance and the development of pre-diabetes. In one embodiment, euglycemia is defined as the condition in which a subject has a fasting blood glucose concentration within the normal range, greater than 70 mg/dl (3.89 mmol/L) and less than 110 mg/dl (6.11 mmol/L). The word fasting has the usual meaning as a medical term. In one embodiment, impaired glucose tolerance (IGT), is defined as the condition in which a subject has a fasting blood glucose concentration or fasting serum glucose concentration greater than 110 mg/dl and less than 126 mg/dl (7.00 mmol/L), or a 2 hour postprandial blood glucose or serum glucose concentration greater than 140 mg/dl (7.78 mmol/L) and less than 200 mg/dl (11.11 mmol/L). The term impaired glucose tolerance is also intended to apply to the condition of impaired fasting glucose. In one embodiment, hyperinsulinemia is defined as the condition in which a subject with insulin resistance, with or without euglycemia, in which the fasting or postprandial serum or plasma insulin concentration is elevated above that of normal, lean individuals without insulin resistance, having a waist-to-hip ration<1.0 (for men) or <0.8 (for women).

In some embodiments, “obesity” refers to a body mass index (BMI) of 30 kg/²m or more (National Institute of Health, Clinical Guidelines on the Identification, Evaluation, and Treatment of Overweight and Obesity in Adults (1998), which is expressly incorporated by reference). However, in some embodiments of the present invention, at least in part, is also intended to include a disease, disorder, or condition that is characterized by a body mass index (BMI) of 25 kg/²m or more, 26 kg/²m or more, 27 kg/²m or more , 28 kg/²m or more, 29 kg/²m or more, 29.5 kg/²m or more, or 29.9 kg/²m or more, all of which are typically referred to as overweight (National Institute of Health, Clinical Guidelines on the Identification, Evaluation, and Treatment of Overweight and Obesity in Adults (1998), which is expressly incorporated by reference). The obesity described herein may be due to any cause, whether genetic or environmental. In one embodiment, “prevention of obesity” refers to preventing obesity or an obesity-associated disorder from occurring if the treatment is administered prior to the onset of the obese condition. Moreover, if treatment is commenced in subjects already suffering from or having symptoms of obesity or an obesity-associated disorder, such treatment is expected to prevent, or to prevent the progression of obesity or the obesity-associated disorder.

The term “obesity-associated disorder” includes all disorders associated with or caused at least in part by obesity. Obesity-associated disorders include, for example, diabetes; cardiovascular disease; high blood pressure; deep vein thrombosis; osteoarthritis; obstructive sleep apnea; cancer and non-alcoholic fatty liver disease.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997), which is incorporated by reference herein. Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus or carboxy-terminus of the reference polypeptide, or alternatively both. Fragments typically are at least 5, 6, 7, 8, 9, or 10 amino acids long, at least 14 amino acids long, at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids long, at least 75 amino acids long, or at least 100, 115, 125, 135, 150, 160, 175, 180, 190, 200, 215, 230, 250, 275, 290, 300, 250, 400, 425, 450, 475, 500 or more amino acids long.

A fragment can retain one or more of the biological activities of the reference polypeptide. In certain embodiments, a fragment may comprise a druggable region, and optionally additional amino acids on one or both sides of the druggable region, which additional amino acids may number from 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or up to 100 or more residues. Further, fragments can include a sub-fragment of a specific region, which sub-fragment retains a function of the region from which it is derived. In another embodiment, a fragment may have immunogenic properties. Fragments may be devoid of about 1, 2, 3, 4, 5, 6, 7, 8, 9, 10, 15, 20, 25, 30, 35, 40, 45, 50, 75, 100 or more amino acids at the N- or C-terminus of the wildtype protein.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays described herein. The term “small organic molecule” refers to a small molecule that is often identified as being an organic or medicinal compound, and does not include molecules that are exclusively nucleic acids, peptides or polypeptides.

As used herein, the terms “subject” and “subjects” refer to an animal, e.g., a mammal including a non-primate (e.g., a cow, pig, horse, donkey, goat, camel, cat, dog, guinea pig, rat, mouse, sheep) and a primate (e.g., a monkey, such as a cynomolgous monkey, gorilla, chimpanzee and a human). In some embodiments, the subject or patient is afflicted with a metabolic disorder such as obesity.

The phrases “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### III. Kallikrein Family Peptidases

As used herein, the term “kallikrein family peptidase” or “kallikrein family member protein” refers to the family of serene proteases whose amino acid sequences are provided in SEQ ID NO: 1-41, as well as biologically active fragments thereof (e.g., fragments of at least 50, 60, 70, 80, 90, 100, 110, 120, 130, 140, 150, 160, 170, 180, 190, 200, 210, 220, 225, 230, 235 or 240 amino acids), and biologically active homologous variants thereof (e.g., proteins having at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity to a sequence provided in SEQ ID NO: 1-41). Exemplary biologically active fragments and/or homologous variants of a kallikrein family member can, for example, have serine protease activity and/or the ability to enhance insulin sensitivity. Variants of kallikrein family member proteins can be produced by standard means, including site-directed and random mutagenesis. In some embodiments the kallikrein related peptidase is Klk1b22 (SEQ ID NO: 38) or a fragment or homologous variant thereof. In some embodiments, the kallikrein related peptidase is Klk1 (SEQ ID NO: 1) or Klk12 (SEQ ID NO: 12) or a fragment or homologous variant thereof.

Kallikrein family member proteins are often translated in an inactive pre-protein form, after which they are proteolytically cleaved into an active protein (see, e.g., Schmaier, International Immunopharmacology 8:161-165 (2008), which is expressly incorporated by reference herein. As used herein, the term “kallikrein family peptidase” and “kallikrein family member protein” encompasses both the inactive pre-protein and the active form of the protein.

The ability of a kallikrein family member protein to enhance insulin sensitivity can be determined either in vivo or in vitro using any method known in the art. For example, to determine the ability of a kallikrein family member protein to enhance insulin sensitivity in vitro, the protein can be added to a cell line being grown in culture and the phosphorylation of the insulin receptor in response to insulin measured over time. To measure the ability of a kallikrein family member protein to enhance insulin sensitivity in vivo, the protein can be injected or expressed in an animal (e.g., a mouse) and glucose clearance in the blood determined after administration of a glucose bolus or an injection of insulin. Non-limiting examples of methods of measuring insulin sensitivity are also provided in the examples below.

In certain embodiments, a protein described herein is further linked to a heterologous polypeptide, e.g., a polypeptide comprising a domain which increases its solubility and/or facilitates its purification, identification, detection, and/or structural characterization. A protein described herein may be linked to at least 2, 3, 4, 5, or more heterologous polypeptides. Polypeptides may be linked to multiple copies of the same heterologous polypeptide or may be linked to two or more heterologous polypeptides. The fusions may occur at the N-terminus of the polypeptide, at the C-terminus of the polypeptide, or at both the N- and C-terminus of the polypeptide. In some embodiments a linker sequence is included between a protein described herein and the fusion domain in order to facilitate construction of the fusion protein or to optimize protein expression or structural constraints of the fusion protein.

In another embodiment, a protein may be modified so that its rate of traversing the cellular membrane is increased. For example, the polypeptide may be fused to a second peptide which promotes “transcytosis,” e.g., uptake of the peptide by cells. The peptide may be a portion of the HIV transactivator (TAT) protein, such as the fragment corresponding to residues 37-62 or 48-60 of TAT, portions which have been observed to be rapidly taken up by a cell in vitro (Green and Loewenstein, (1989) Cell 55:1179-1188). Alternatively, the internalizing peptide may be derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is coupled. Thus, the polypeptide may be fused to a peptide consisting of about amino acids 42-58 of Drosophila antennapedia or shorter fragments for transcytosis (Derossi et al. (1996) J Biol Chem 271:18188-18193; Derossi et al. (1994) J Biol Chem 269:10444-10450; and Perez et al. (1992) J Cell Sci 102:717-722), all of which are incorporated by reference. The transcytosis polypeptide may also be a non-naturally-occurring membrane-translocating sequence (MTS), such as the peptide sequences disclosed in U.S. Pat. No. 6,248,558, which is incorporated by reference.

### IV. Inducers of Kallikrein Family Peptidase Expression

Certain methods described herein relate to the administration of an agent that increases or decreases the activity and/or expression of a kallikrein family peptidase. Agents which may be used to increase the expression or activity of a kallikrein family peptidase include antibodies (e.g., conjugated antibodies), proteins, peptides, small molecules and inhibitory RNA molecules, e.g., siRNA molecules, shRNA, ribozymes, and antisense oligonucleotides. Such agents can be those described herein, those known in the art, or those identified through routine screening assays (e.g. the screening assays described herein). For example, in some embodiments the agent is an inhibitory RNA molecule (e.g., an siRNA or shRNA molecule) specific for RheB GTPase.

In some embodiments, an assay is used to identify agents useful in the methods described herein. For example, provided herein are methods for determining whether a test compound is a likely therapeutic agent for increasing insulin sensitivity, increasing brown fat levels, treating diabetes, treating a metabolic disorder or treating obesity. In general, such methods include the steps of (a) contacting a cell with the test compound; and (b) detecting the expression of a kallikrein family peptidase (e.g., Klk1b22, Klk1 or Klk12) by the cell. A test compound that causes increased expression of a kallikrein family peptidase (for example, compared to cells treated with a placebo or untreated cells) is a likely therapeutic agent.

Any cell can be used in the above described screening method. For example, in some embodiments the cell is a mouse cell or a human cell. In certain embodiments the cell is a T cell or a T cell line. Cells used in the screen can be primary cells or a cell line. Examples of other cell lines useful in the screening assays described herein include, but are not limited to, 293-T cells, 3T3 cells, 721 cells, 9L cells, A2780 cells, A172 cells, A253 cells, A431 cells, CHO cells, COS-7 cells, HCA2 cells, HeLa cells, Jurkat cells, NIH-3T3 cells and Vero cells.

The expression of the kallikrein family peptidase can be detected using any method known in the art. For example, the expression of the kallikrein family peptidase can be detected by detecting kallikrein family peptidase mRNA using, e.g., a detectably labeled nucleic acid probe, RT-PCR, and/or microarray technology. The expression of the kallikrein family peptidase can also be detected by detecting kallikrein family peptidase protein using, e.g., detectably labeled antibodies that with binding specificity for the kallikrein family protease.

In some embodiments, a cell is used in the screening assay that has been genetically engineered to facilitate the performance of the assay. For example, in some embodiments, the cell is engineered such that the kallikrein family peptidase is expressed as a heterologous protein linked to a detectable moiety (e.g. a fluorescent moiety such as GFP or a luminescent moiety such as luciferase). In other embodiments, the cell contains a nucleic acid sequence encoding a detectable moiety operably linked to a kallikrein family member promoter. In such embodiments, rather than detecting expression of the kallikrein family peptidase, the expression of the detectable moiety is detected directly. Such cells can be generated using standard recombinant techniques well known in the art.

Agents useful in the methods of the present invention may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85, which is incorporated by reference); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145, which is incorporated by reference).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233, all of which are incorporated by reference herein.

### V. Recombinant Cells with Reduced Rheb Activity or Expression

Certain embodiments described herein relate to the administration of one or more recombinant cells that have reduced Rheb expression or activity. In certain embodiments, the reduced level of Rheb expression or activity is in comparison to a non-recombinant cell of the same type and species as the recombinant cell. For example, the recombinant cell may be a recombinant human T cell that has reduced Rheb expression compared to a non-recombinant human T cell. In certain embodiments, the recombinant cell and the non-recombinant cell to which the comparison is made are from the same organism (e.g., the same person). In certain embodiments, the Rheb expression or activity of the recombinant cell is 75%, 50%, 40%, 35%, 50%, 25%, 20%, 15%, 10%, 5%, 4%, 3%, 2% or 1% of the Rheb expression of the non-recombinant cell. The level or Rheb expression can be determined, for example, using methods known in the art, such as quantitative RT-PCR or western blotting. In some embodiments, the recombinant cell has no Rheb expression or activity.

The level of Rheb expression in the recombinant cell can be reduced using recombinant DNA technologies known in the art. For example, in certain embodiments one or both of the Rheb genes and/or Rheb promoters in a cell are mutated or knocked out to generate the recombinant cells. In some embodiments an Rheb specific inhibitory RNA expression vector is introduced into the recombinant cells, causing them to express a Rheb-specific inhibitory RNA molecule.

In some embodiments, the recombinant cell is an immune cell or an immune cell precursor, such as a lymphocyte or lymphocyte precursor. For example, in some embodiments the recombinant cells are T cells, T cell precursors, B cells, B cell precursors, bone marrow stem cells, embryonic stem cells, induced embryonic stem cells, peripheral blood stem cells or a combination thereof.

In some embodiments, the recombinant cells are syngeneic to the subject to whom they are to be administered. Recombinant cells that are syngeneic to the subject can be generated, for example, by using recombinant genetic technology to convert non-recombinant cells harvested from the subject into recombinant cells with reduced Rheb expression or activity.

### VI. Pharmaceutical Compositions

The agents described herein (e.g. kallikrein family peptidases, cells that have reduced Rheb activity or expression and/or agents that increase kallikrein family peptidase expression or activity) can be incorporated into pharmaceutical compositions suitable for administration to a subject. The compositions may contain a single such agent or any combination of modulatory agents described herein and a pharmaceutically acceptable carrier. The pharmaceutical composition may further comprise additional agents useful for treating diabetes or metabolic disorders, such as obesity.

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral, transdermal (topical), transmucosal, and rectal administration.

Toxicity and therapeutic efficacy of the agents described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods described herein of the invention, the therapeutically effective dose can be estimated initially from cell culture assays. A dose can be formulated in animal models to achieve a circulating plasma concentration range that includes the 1050 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

Appropriate doses agents depends upon a number of factors within the scope of knowledge of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

### VII. Therapeutic Methods

In some embodiments, the present invention relates to a method for increasing insulin sensitivity, preventing or treating diabetes (e.g., type I or type II diabetes), increasing brown fat levels, preventing or treating a metabolic disorder (e.g., obesity), promoting weight loss, treating hyperlipidemia and/or treating cardiovascular and vascular disease by administering to a subject (e.g. a subject in need thereof) an agent described herein (e.g. a kallikrein family peptidase, a recombinant cell that has reduced Rheb expression or activity, or an agent that increases expression of a kallikrein family peptidase.

A subject in need thereof may include, for example, a subject who has been diagnosed with diabetes, a subject who has been diagnosed with a metabolic disease, a subject who is obese, or a subject who has been treated for a metabolic disease or diabetes, including subjects that have been refractory to previous treatment. A subject in need thereof may also include, for example, a subject that is predisposed to metabolic diseases or diabetes, including subjects who are predisposed to obesity, subjects who are overweight and subjects with a family history of metabolic disease or diabetes.

In certain embodiments, the methods described herein relate to the treatment of diabetes. The methods described herein can be used to treat any form of diabetes, including type I diabetes, type II diabetes and gestational diabetes.

In certain embodiments, the methods described herein encompass the treatment of any metabolic disorder. In certain embodiments, the metabolic disorder treated is obesity, insulin resistance, hyperinsulinemia, hypoinsulinemia, type II diabetes, hypertension, hyperhepatosteatosis, hyperuricemia, fatty liver, non-alcoholic fatty liver disease, polycystic ovarian syndrome, acanthosis nigricans, hyperphagia, endocrine abnormalities, triglyceride storage disease, Bardet-Biedl syndrome, Lawrence-Moon syndrome, Prader-Labhart-Willi syndrome, or muscle hypoplasia. In certain embodiments the metabolic disorder is an obesity-associated disorder, such as diabetes, cardiovascular disease, high blood pressure, deep vein thrombosis, osteoarthritis, obstructive sleep apnea, cancer or non-alcoholic fatty liver disease

In some embodiments, the pharmaceutical composition described herein will incorporate the therapeutic agent to be delivered in an amount sufficient to deliver to a patient a therapeutically effective amount of an incorporated therapeutic agent or other material as part of a prophylactic or therapeutic treatment. The desired concentration of the active agent will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of the subject agent may be determined by reference to the plasma concentrations of the agent. For example, the maximum plasma concentration (Cmax) and the area under the plasma concentration-time curve from time 0 to infinity (AUC (0-4)) may be used. Dosages for the present invention include those that produce the above values for Cmax and AUC (0-4) and other dosages resulting in larger or smaller values for those parameters.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the agents of the invention employed in the pharmaceutical composition at levels lower than that required in order to achieve the desired therapeutic effect and gradually increase the dosage until the desired effect is achieved.

In general, a suitable daily dose of an agent described herein will be that amount of the agent which is the lowest dose effective to produce a therapeutic effect. Such an effective dose will generally depend upon the factors described above.

In certain embodiments, the kallikrein family peptidase is administered to the subject by administering a population of cells that expresses the kallikrein family peptidase to the subject. In certain embodiments, the cells are engineered to express elevated levels of the kallikrein family peptidase. For example, standard recombinant technology can be used to insert into the cells a transgene containing a kallikrein family peptidase encoding nucleic acid operably linked to either a constitutive or conditional promoter. Alternatively, the cell can be engineered so that it expresses reduced levels of Rheb GTPase, either by knocking out the endogenous Rheb GTPase genes or by inserting a transgene that contains a Rheb GTPase inhibitory RNA molecule encoding nucleic acid operably linked to either a constitutive or conditional promoter.

In certain embodiments, the population of kallikrein family peptidase expressing cells administered to the subject contains T cells, T cell precursors, B cells, B cell precursors, bone marrow stem cells, embryonic stem cells, induced embryonic stem cells, peripheral blood stem cells or a combination thereof. In certain embodiments, the subjects own cells are used to generate the population of kallikrein expressing cells. Fore example, in some embodiments bone marrow or peripheral blood stem cells are isolated from the subject, engineered to express elevated levels of a kallikrein family peptidase as described above, and then administered back to the subject. In such embodiments, rejection of the cells is less likely.

This invention is further illustrated by the following examples which should not be construed as limiting. The contents of all references, patents and published patent applications cited throughout this application, as well as the Figures, are incorporated herein by reference.

### EXAMPLE 1

The mTOR signaling pathway is a nutrient-sensing cascade that plays a critical role in regulating cell proliferation and survival. A line of mice that lack a member of this signaling cascade, Rheb, only in the T cells was developed using LoxP-cre technology (hereby referred to as Rheb^{fl/fl} CD4cre).

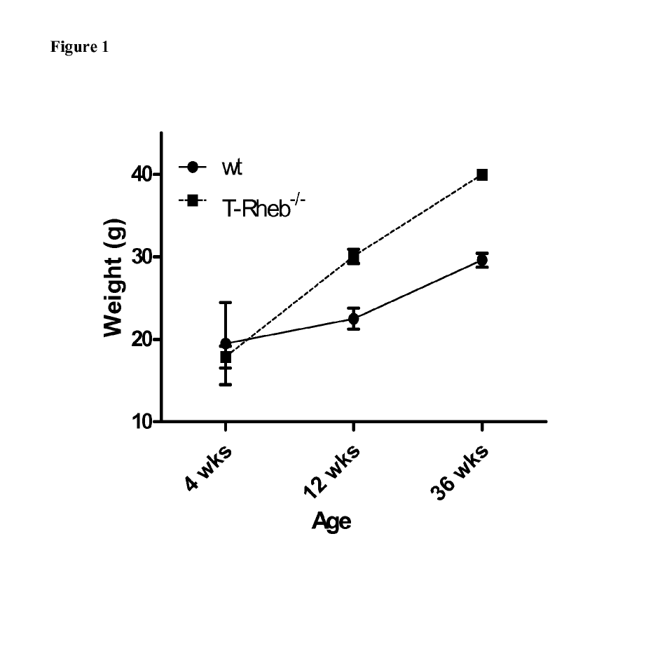

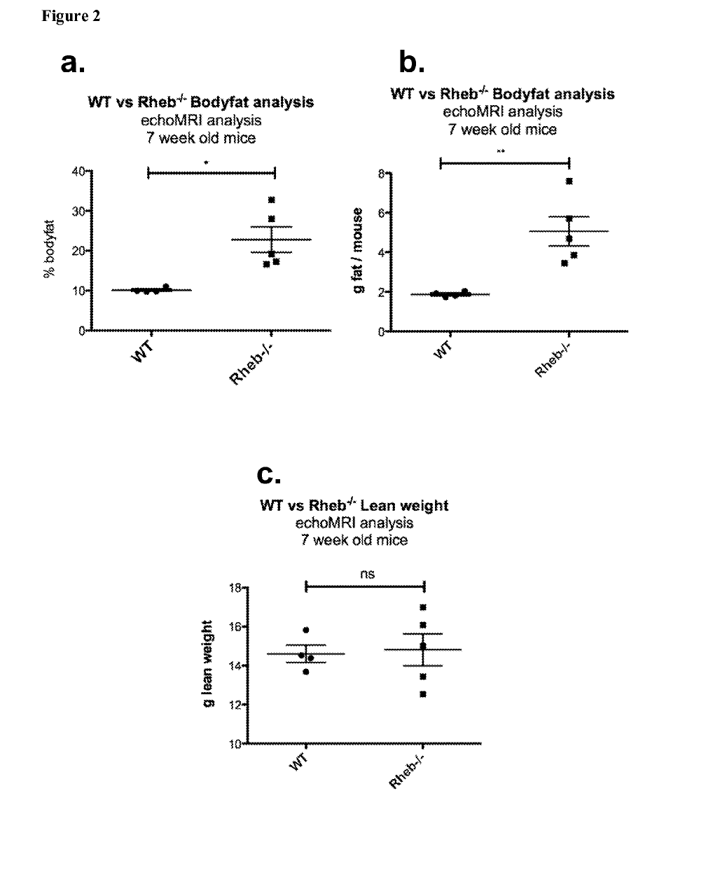

The Rheb^{fl/fl} CD4cre mice gained weight at a faster rate than their WT counterparts (FIG. 1). These mice showed a substantial increase in the amount of body fat deposition, with a 7 week old Rheb^{fl/fl} CD4cre mouse exhibiting 3-6 times the mass of body fat compared to their WT counterparts (FIG. 2a, 2b). There was no increase in lean mass body mass in the Rheb^{fl/fl} CD4cre mice (FIG. 2c).

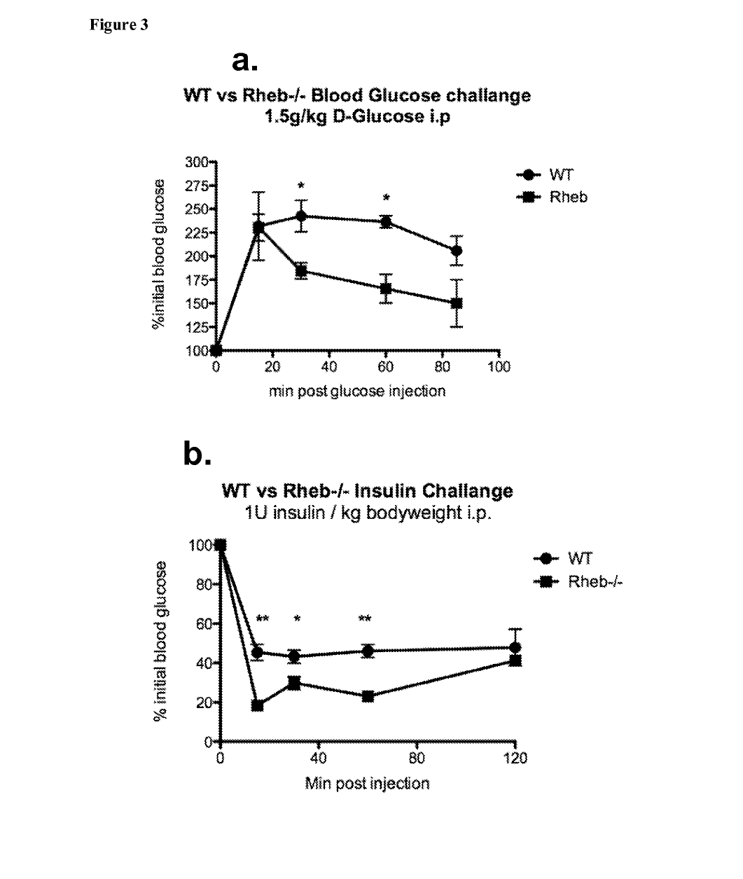

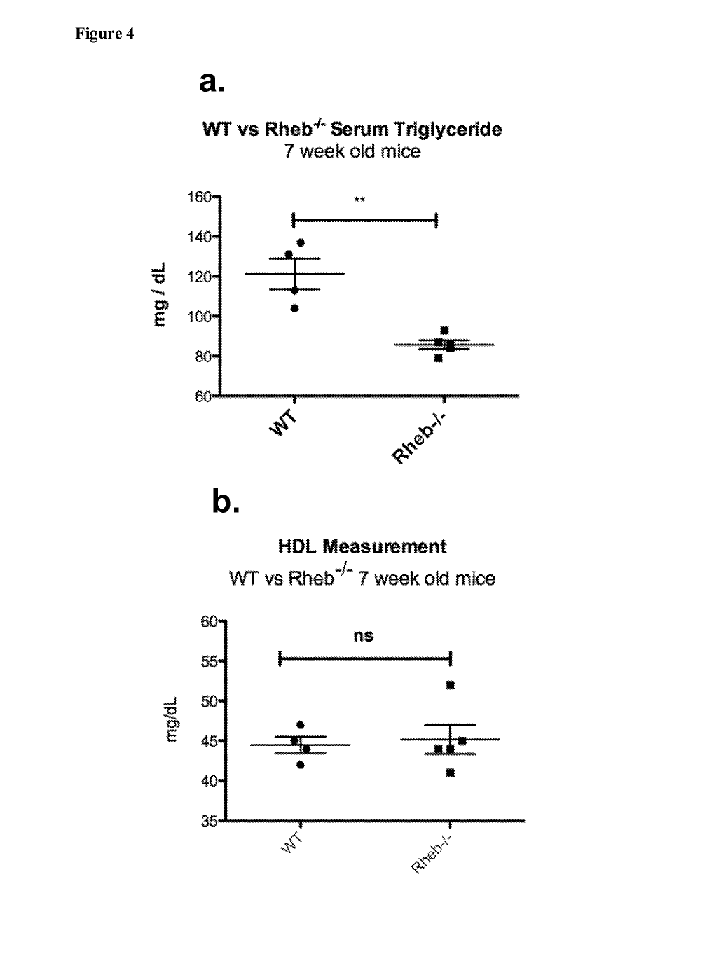

When the ability of the Rheb^{fl/fl} CD4cre mice to uptake glucose was investigated, it was observed that they exhibited an increased ability to uptake glucose from their bloodstream following intraperitoneal D-Glucose challenge (FIG. 3a), and increased insulin sensitivity (FIG. 3b). It was also observed that the Rheb^{fl/fl} CD4cre mice have lower serum triglyceride levels than WT controls (FIG. 4a), but no difference in serum High-density lipoprotein (HDL) levels (FIG. 4b). This is a phenotype traditionally associated with a “fit” condition, and not that normally found in severely obese individuals.

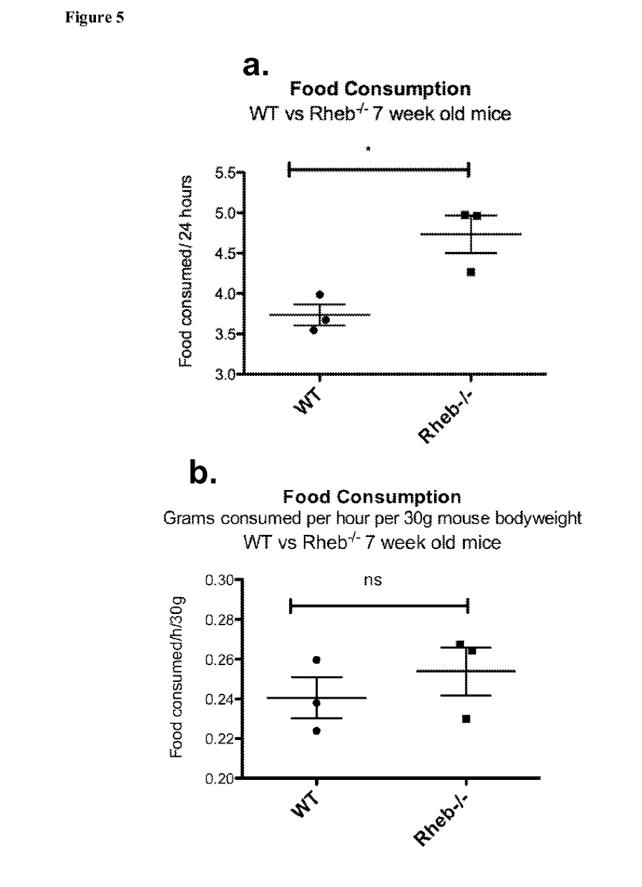

While Rheb^{fl/fl} CD4cre mice ate more food in a 24 hr period than age-matched WT controls (FIG. 5a), there was no difference in the amount of food eaten per hour per 30 g of bodyweight (FIG. 5b), suggesting that the increase in body weight was not due to a neurological disorder, but rather some difference in nutrient metabolism.

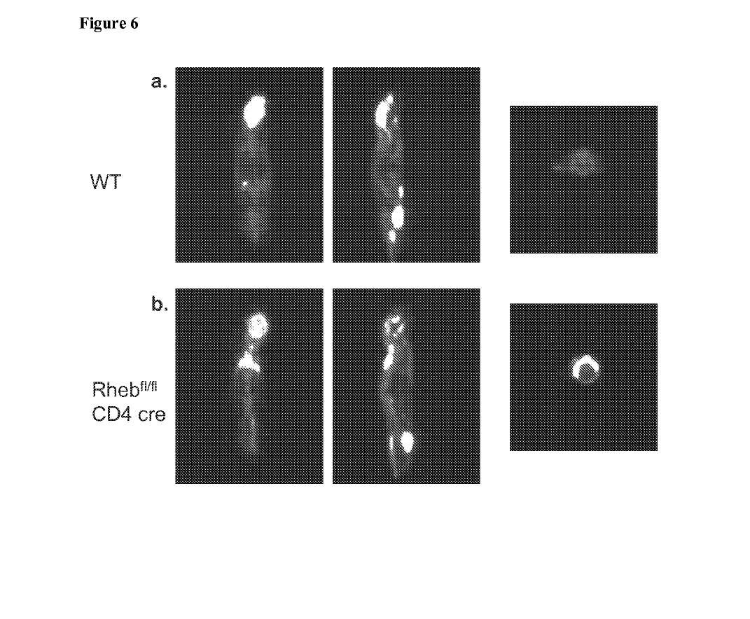

To determine which tissue was responsible for the enhanced glucose uptake and increased insulin sensitivity of the Rheb^{fl/fl} CD4cre mice, 18F-FDG Proton Emission Tomography (PET) imaging was used to visualize tissue uptake of radioactive glucose. In WT mice, the majority of the detectible signal localized to the bladder, brain, heart and intestine (FIG. 6a). However, in the Rheb^{fl/fl} CD4cre mice there was a high level of glucose uptake in the tissue on the back between the shoulder blades (FIG. 6b). This anatomical location and metabolic profile suggests that Rheb^{fl/fl} CD4cre mice have significant deposits of Brown Adipose Tissue (BAT) compared to WT controls. BAT is known to be highly vascular and to possess a high rate of glucose metabolism.

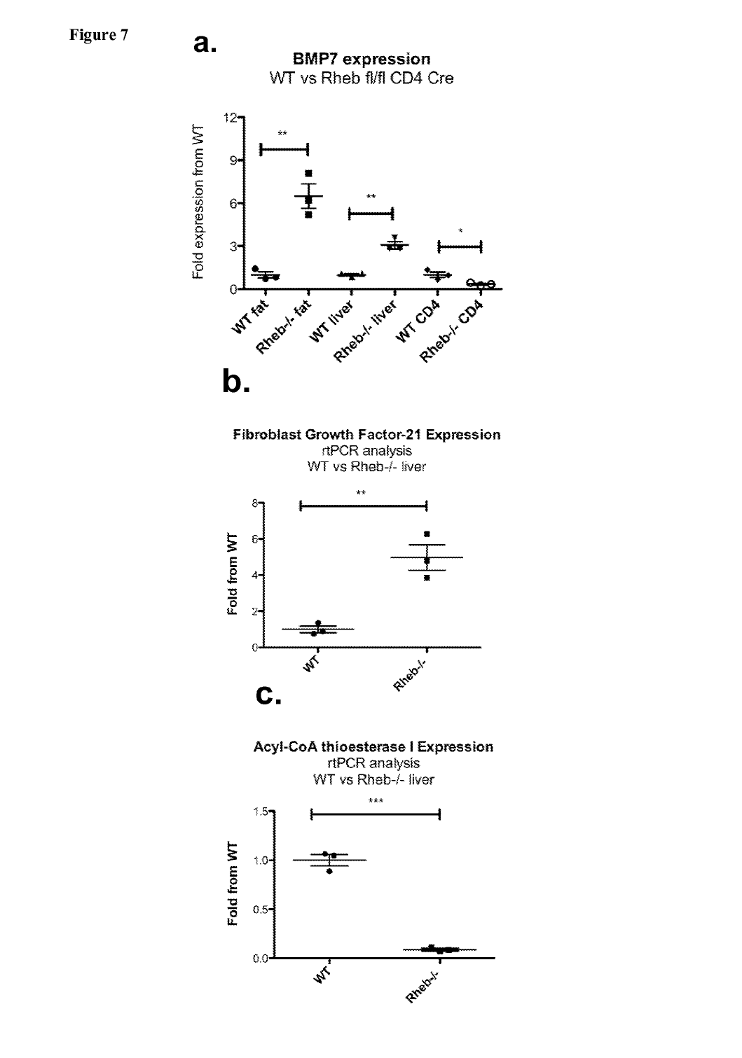

Real time PCR analysis of liver and adipose tissue was performed detect a gene expression profile that would explain the phenotype observed in Rheb^{fl/fl} CD4cre mice. Increased in the expression of Bone Morphogenic Protein 7 (BMP-7), a member of the TGF-β family that potentiates the development of Brown Adipose Tissue, was observed in the liver of Rheb^{fl/fl} CD4cre mice (FIG. 7a). Increased expression of Fibroblast Growth Factor 21 (FGF-21) was also observed in the Rheb^{fl/fl} CD4cre mice. FGF-21 is a factor that stimulates glucose uptake in adipose tissue (FIG. 7b). Finally, decreases expression of acyl-CoA thioesterase I was observed in the livers of Rheb^{fl/fl} CD4cre mice (FIG. 7c).

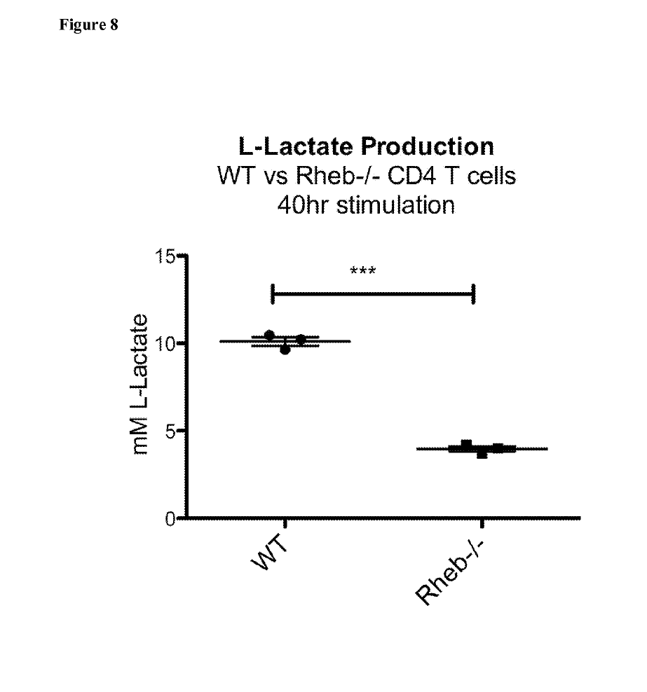

The ability of T cells from Rheb^{fl/fl} CD4cre mice to utilize glucose was examined. CD4 T cells isolated from Rheb^{fl/fl} CD4cre mice have a lower rate of glycolysis than their WT counterparts, as measured by lactate acid production (FIG. 8).

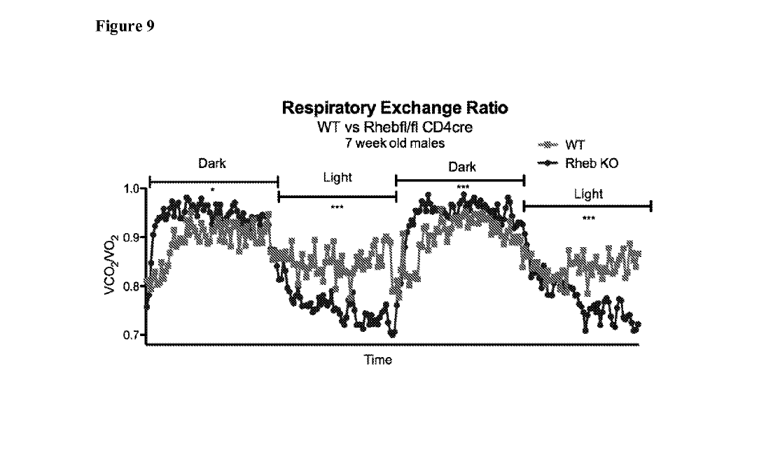

Oxymax Indirect calorimetry was used to measure the Respiratory Exchange Ratio (RER) in WT and Rheb^{fl/fl} CD4cre mice. This technique determines the nature of the metabolic substrate used by the mouse, with a score near 1 indicating carbohydrate utilization, and a score near 0.7 indicating fat utilization. The Rheb^{fl/fl} CD4cre mouse showed dramatic swings in RER scores during the light/dark cycles (FIG. 9). These results suggest that during the dark cycle, when food is consumed, the Rheb^{fl/fl} CD4cre mice primarily utilize the carbohydrates consumed in their chow for energy production. However, during the light cycle, where little food is consumed, they rely on fat stores. These results are consistent with the hyper-insulin sensitivity of the Rheb^{fl/fl} CD4cre mice.

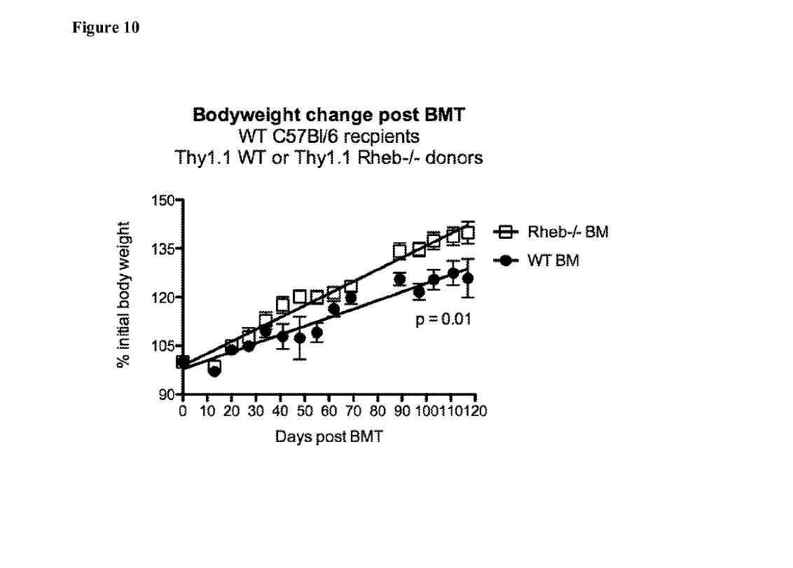

To ensure that the phenotype that observed in the Rheb^{fl/fl} CD4cre mice was not due to an ectopic deletion of Rheb in tissue other than the T cells, a Bone Marrow Transplant (BMT) performed using bone marrow isolated from WT and Rheb^{fl/fl} CD4cre mice and injected into irradiated WT recipients. As with the parental strain, mice receiving bone marrow from Rheb^{fl/fl} CD4cre mice gained weight faster than mice receiving WT bone marrow (FIG. 10).

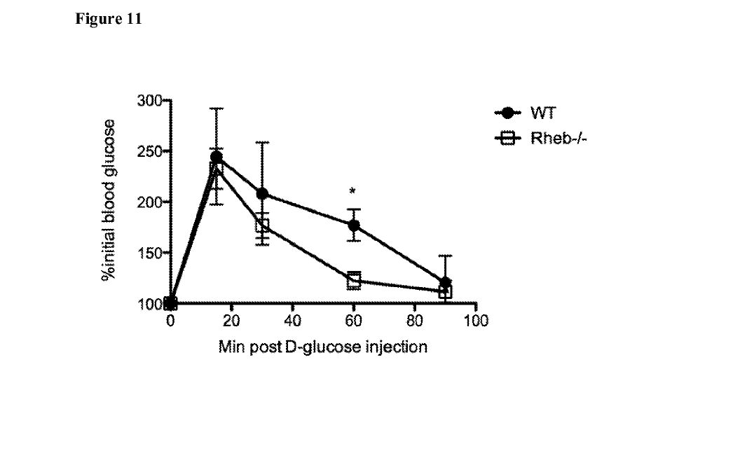

Next, CD4 T cells were transferred from WT and Rheb^{fl/fl} CD4cre mice into Rag−/− recipients. After 2.5 weeks, the mice were fasted for 6 hours and their ability to remove glucose from the bloodstream was tested. As shown in FIG. 11, mice receiving CD4 T cells from Rheb^{fl/fl} CD4cre mice showed an enhancement in their ability to remove glucose from the bloodstream. This demonstrates that it is possible to modulate global glucose sensitivity in a host simply by providing a CD4 T cell population lacking Rheb activity.

A microarray analysis of CD4 and CD8 T cells isolated from WT and Rheb^{fl/fl} CD4cre mice was performed. The gene Klk1b22, a member of the kallikrein protease family, was very highly expressed in both the CD4 and CD8 cells isolated from a Rheb^{fl/fl} CD4cre mice. This result was confirmed by RT-PCR.

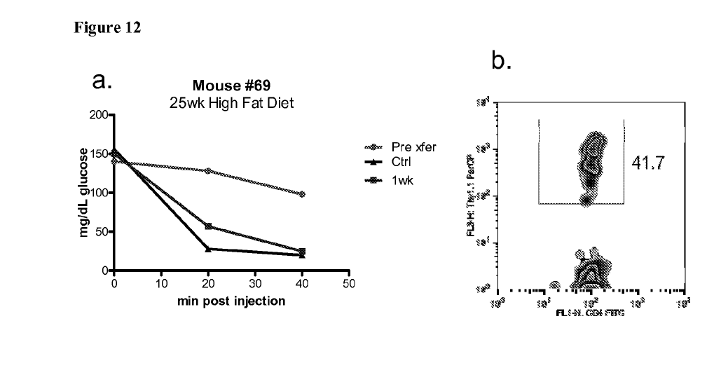

It was next tested whether the adoptive transfer of Rheb deficient T cells could reverse the insulin insensitivity induced by a prolonged high-fat diet. To this end, WT mice were maintained on a 60% fat diet for 25 weeks, after which point ˜40% of the mice exhibited significant insulin insensitivity. At this point, the diabetic mice were lightly irradiated (700rad) and injected i.v. with 4×10⁶ CD4 T cells isolated from Rheb^{fl/fl} CD4cre mice. After 1 week the diabetic mice were bled to determine the level of expansion of the adoptively transferred T cells, and their sensitivity to insulin determined. Approximately 50% of the mice receiving the Rheb^{fl/fl} CD4cre T cell transfer displayed an increased sensitivity to insulin (FIG. 12a). Furthermore, the mice with the most robust increase in insulin sensitivity were those that had the highest degree of reconstitution by the donor T cells (FIG. 12b). Thus transfer of Rheb^{fl/fl} CD4cre T cells into type II diabetic mice was able to cure them of their diabetes.

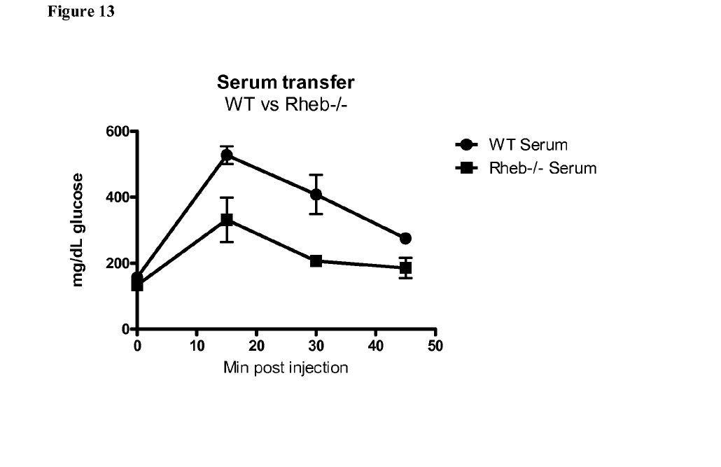

The ability of the serum of Rheb^{fl/fl} CD4cre mice to convey enhanced glucose uptake was tested. WT and Rheb^{fl/fl} CD4cre mice were fasted for 2 hours, then bled by cardiac puncture and their serum isolated. 100 μl of serum was injected i.v. into WT mice that had been fasted for 4 hours. After 30 min, recipient mice were injected i.p. with 1.5 g/kg D-glucose. Blood glucose levels were measured using a OneTouch Ultra system. As shown in FIG. 13, mice administered serum from Rheb^{fl/fl} CD4cre mice had significantly elevated levels of glucose uptake compared to mice administered serum from WT mice.

### EXAMPLE 2

Klk1b22 has further been determined herein to enhance both insulin receptor phosphorylation and prolong insulin receptor phosphorylation in response to a dose response of insulin. Specifically, HEK293T cells were incubated with insulin at different doses in the presence or absence of recombinant (Flag tag-isolated) Klk1b22. Lysates were collected and insulin receptor phosphorylation was assessed by Western blot analysis (FIG. 18A). Similarly, HEK293T cells were incubated in the presence or absence of recombinant (Flag tag-isolated) Klk1b22. Lysates were collected at different time points and insulin receptor phosphorylation was assessed by Western blot analysis (FIG. 18B). The data shown in FIGS. 18A-18B demonstrate that Klk1b22 enhances both insulin receptor phosphorylation and prolong insulin receptor phosphorylation in response to a dose response of insulin.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for preventing or treating diabetes in a subject comprising administering to the subject a Kallikrein family peptidase or biologically active fragment thereof

2. The method of claim 1, wherein the Kallikrein family peptidase or biologically active fragment thereof has an amino acid sequence that is at least 70% identical to a sequence selected from the group consisting of SEQ ID NO: 1-41.

3. The method of claim 1, wherein the Kallikrein family peptidase or biologically active fragment thereof has an amino acid sequence that is at least 70% identical to SEQ ID NO: 38.

4. The method of claim 1, wherein the Kallikrein family peptidase or biologically active fragment thereof has an amino acid sequence that is at least 70% identical to SEQ ID NO: 12.

5. The method of claim 1, wherein the Kallikrein family peptidase or biologically active fragment thereof has an amino acid sequence that is at least 70% identical to SEQ ID NO: 1.

6. The method of claim 1, wherein the diabetes is Type I diabetes.

7. The method of claim 1, wherein the diabetes is Type II diabetes.

8. The method of claim 1, wherein the Kallikrein family peptidase or biologically active fragment thereof is administered by administering to the subject a pharmaceutical composition comprising an isolated Kallikrein family peptidase or biologically active fragment thereof

9. The method of claim 1, wherein the Kallikrein family peptidase or biologically active fragment thereof is administered by administering to the subject a population of recombinant cells that expresses the Kallikrein family peptidase or a biologically active fragment thereof.

10. The method of claim 9, wherein the population of recombinant cells comprise T cells, T cell precursors, B cells, B cell precursors, bone marrow stem cells, embryonic stem cells, induced embryonic stem cells, peripheral blood stem cells or a combination thereof

11. A method for increasing insulin sensitivity in a subject comprising administering to the subject a Kallikrein family peptidase or biologically active fragment thereof.

12. The method of claim 11, wherein the Kallikrein family peptidase or biologically active fragment thereof has an amino acid sequence that is at least 70% identical to a sequence selected from the group consisting of SEQ ID NO: 1-41.

13-17. (canceled)

18. The method of claim 11, wherein the Kallikrein family peptidase or biologically active fragment thereof is administered by administering to the subject a pharmaceutical composition comprising an isolated Kallikrein family peptidase or biologically active fragment thereof

19. The method of claim 11, wherein the Kallikrein family peptidase or biologically active fragment thereof is administered by administering to the subject a population of recombinant cells that expresses the Kallikrein family peptidase or a biologically active fragment thereof

20. (canceled)

21. A method for preventing or treating a metabolic disorder in a subject comprising administering to the subject a Kallikrein family peptidase or biologically active fragment thereof

22. The method of claim 21, wherein the Kallikrein family peptidase or biologically active fragment thereof has an amino acid sequence that is at least 70% identical to a sequence selected from the group consisting of SEQ ID NO: 1-41.

23-25. (canceled)

26. The method of claim 21, wherein the metabolic disorder is obesity.

27. The method of claim 21, wherein the Kallikrein family peptidase or biologically active fragment thereof is administered by administering to the subject a pharmaceutical composition comprising an isolated Kallikrein family peptidase or biologically active fragment thereof

28. The method of claim 21, wherein the Kallikrein family peptidase or biologically active fragment thereof is administered by administering to the subject a population of recombinant cells that expresses the Kallikrein family peptidase or a biologically active fragment thereof

29. The method of claim 28, wherein the population of recombinant cells comprise T cells, T cell precursors, B cells, B cell precursors, bone marrow stem cells, embryonic stem cells, induced embryonic stem cells, peripheral blood stem cells or a combination thereof

30-94. (canceled)
